(19) United States
(12) Patent Application Publication
(10) Pub. No.: US 2026/0110661 A1
(43) Pub. Date: Apr. 23, 2026

(54) GAS SENSOR

(71) Applicant: NGK INSULATORS, LTD., Nagoya-shi (JP)

(72) Inventors: Taku OKAMAOTO, Nagoya-shi (JP); Daichi ICHIKAWA, Nagoya-shi (JP)

(21) Appl. No.: 19/424,233

(22) Filed: Dec. 18, 2025

Related U.S. Application Data
(63) Continuation of application No. PCT/JP2024/016427, filed on Apr. 26, 2024.

(30) Foreign Application Priority Data
Jul. 3, 2023 (JP) 2023-109462

(51) Int. Cl. G01N 27/417; G01N 27/30; G01N 27/409; G01N 27/41; G01N 27/419; G01N 27/49; G01N 33/00

(52) U.S. Cl. CPC G01N 27/4175; G01N 27/301; G01N 27/41; G01N 27/419; G01N 27/49; G01N 33/004; G01N 27/409

(57) ABSTRACT

A gas sensor includes a sensor element and a control device, and configured to measure a carbon dioxide concentration in a measurement gas, wherein: the sensor element includes an element body, a first pump cell, a second pump cell, a third pump cell including a third inner electrode, a reference electrode, and the control device performs a third inner electrode degradation determination processing for determining degradation of the third inner electrode based on a third voltage between third inner electrode and reference electrode during execution of first and second pump cell control processing and during stoppage of third pump cell control processing, and based on the third pump current flowing through the third pump cell during execution of the first to third pump cell control processing.

### CROSS-REFERENCE TO RELATED APPLICATION

This application is a continuation application of PCT/JP2024/016427, filed on Apr. 26, 2024, which claims the benefit of priority from Japanese Patent Application No. 2023-109462, filed on Jul. 3, 2023, the entire contents of which are incorporated herein by reference.

### BACKGROUND OF THE INVENTION

### 1. Field of the Invention

The present invention relates to a gas sensor.

### 2. Description of the Related Art

Hitherto, gas sensors that measure a water concentration and a carbon dioxide concentration in a measurement gas such as exhaust gas from an automobile have been known. For example, PTL 1 describes a gas sensor comprising a sensor element having an oxygen-ion-conductive solid electrolyte layer and provided with a gas flow path therein, which measures the concentrations of water vapor component and carbon dioxide component in the measurement gas. The gas flow path is formed so that a gas inlet, a first diffusion rate-limiting section, a first internal cavity, a second diffusion rate-limiting section, and a second internal cavity are communicated in this order. A main pump cell is configured including a main inner pump electrode disposed in the first internal cavity and an outer pump electrode disposed on an outer surface of the sensor element. A first measurement pump cell is configured including a first measurement inner pump electrode disposed in the second internal cavity and the outer pump electrode. A second measurement pump cell is configured including a second measurement inner pump electrode disposed on the opposite side of the second diffusion rate-limiting section with respect to the first measurement inner pump electrode, and the outer pump electrode. In this gas sensor, an oxygen partial pressure in the first internal cavity is adjusted by the main pump cell so that substantially all of the water vapor component and the carbon dioxide component in the measurement gas are decomposed in the first internal cavity. Then, oxygen is supplied to the second internal cavity by the first measurement pump cell so that hydrogen generated by a decomposition of the water vapor component is selectively burned (oxidized) in the second internal cavity, and the concentration of the water vapor component present in the measurement gas is measured based on a magnitude of a current flowing then. In addition, oxygen is supplied to a vicinity of a surface of the second measurement inner pump electrode by the second measurement pump cell so that carbon monoxide generated by a decomposition of the carbon dioxide component is selectively burned (oxidized) in the vicinity of the surface of the second measurement inner pump electrode, and the concentration of the carbon dioxide component present in the measurement gas is measured based on a magnitude of a current flowing then.

### CITATION LIST

### Patent Literature

PTL 1: JP 5918177 B

### SUMMARY OF THE INVENTION

In such a gas sensor, the third inner electrode (the second measurement inner pump electrode in PTL 1) for oxidizing carbon monoxide may degrade with use of the gas sensor, and an accuracy of measurement of the carbon dioxide concentration may decrease.

The present invention was made to solve such a problem, and its main object is to determine degradation of the third inner electrode.

The present invention employs the following configuration to achieve the above-described main object.
[1] A gas sensor according to the present invention is a gas sensor including a sensor element and a control device, and configured to measure a carbon dioxide concentration in a measurement gas, wherein: the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on an outer surface of the element body; a second pump cell including a second inner electrode disposed in a second chamber located downstream of the first chamber of the measurement gas flow path, and a second outer electrode disposed on an outer surface of the element body; a third pump cell including a third inner electrode disposed in a third chamber located downstream of the second chamber of the measurement gas flow path, and a third outer electrode disposed on an outer surface of the element body; and a reference electrode disposed inside the element body so as to be in contact with a reference gas; the control device performs: a first pump cell control processing in which oxygen is pumped out from around the first inner electrode to around the first outer electrode by controlling the first pump cell, thereby reducing water and carbon dioxide in the measurement gas in the first chamber; a second pump cell control processing in which oxygen is pumped from around the second outer electrode to around the second inner electrode by controlling the second pump cell, thereby oxidizing hydrogen generated by the reduction of water in the first chamber in the second chamber; a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and a carbon dioxide concentration measurement processing for measuring the carbon dioxide concentration in the measurement gas based on a third pump current flowing through the third pump cell by the third pump cell control processing; the control device performs a third inner electrode degradation determination processing for determining degradation of the third inner electrode based on a third voltage between the third inner electrode and the reference electrode during execution of the first and second pump cell control processing and during stoppage of the third pump cell control processing, and based on the third pump current flowing through the third pump cell during execution of the first to third pump cell control processing.

In this gas sensor, the control device performs a third inner electrode degradation determination processing for determining degradation of the third inner electrode based on a third voltage between the third inner electrode and the reference electrode during execution of the first and second pump cell control processing and during stoppage of the third pump cell control processing, and based on the third pump current flowing through the third pump cell during execution of the first to third pump cell control processing. Here, both the above-described third voltage and third pump current take values corresponding to the carbon dioxide concentration in the measurement gas. However, the third pump current is more likely than the third voltage to change its value under the influence of degradation of the third inner electrode, and when the third inner electrode degrades, a correspondence relationship between the third voltage and the third pump current changes. Therefore, the degradation of the third inner electrode can be determined based on the third voltage and the third pump current. The inventors have confirmed this through experiments and analysis.
[2] In the above gas sensor (the gas sensor described in [1]), the control device, in the third inner electrode degradation determination processing, may measure the third voltage and thereafter measures the third pump current. Here, the third pump current has higher responsiveness compared to the third voltage, that is, the time required for it to reach a value corresponding to the concentration of carbon monoxide in the third chamber (a value corresponding to the carbon dioxide concentration in the measurement gas) is shorter. Therefore, by measuring the third voltage first and then measuring the third pump current, the time difference between the two measurements can be reduced. As a result, it is possible to suppress a decrease in the accuracy of determining the degradation of the third inner electrode due to a change in the carbon dioxide concentration of the measurement gas occurring between the two measurements.
[3] In the above gas sensor (the gas sensor described in [1] or [2]), the control device, in the third inner electrode degradation determination processing, may be configured not to perform a determination of degradation of the third inner electrode when the absolute value of the third voltage falls within a predetermined low-voltage region. Here, when the absolute value of the third voltage is relatively low, in other words, when the carbon dioxide concentration in the measurement gas is relatively low, the value of the third pump current is less likely to change even if the third inner electrode is degraded. Therefore, by not performing the determination of the degradation of the third inner electrode when the absolute value of the third voltage falls within the predetermined low-voltage region, the processing load of the control device can be reduced.
[4] In the above gas sensor (the gas sensor described in any one of [1] to [3]), the control device, in the third inner electrode degradation determination processing, may perform a determination of degradation of the third inner electrode based on a difference between the third pump current derived from a measured value of the third voltage and a measured value of the third pump current; or may perform a determination of degradation of the third inner electrode based on a difference between a carbon dioxide concentration derived from a measured value of the third voltage and a carbon dioxide concentration derived from a measured value of the third pump current; or may perform a determination of degradation of the third inner electrode based on a difference between a measured value of the third voltage and the third voltage derived from a measured value of the third pump current.
[5] In the above gas sensor (the gas sensor described in any one of [1] to [4]), the control device, in the carbon dioxide concentration measurement processing, may derive the carbon dioxide concentration based on a corrected third pump current obtained by correcting the third pump current taking into account a result of the third inner electrode deterioration determination processing, or may derive the carbon dioxide concentration by correcting a provisional carbon dioxide concentration based on the third pump current taking into account a result of the third inner electrode deterioration determination processing. In this way, by correcting, it is possible to suppress a decrease in the measurement accuracy of the carbon dioxide concentration due to degradation of the third inner electrode.
[6] In the above gas sensor (the gas sensor described in [4]), the control device, in the carbon dioxide concentration measurement processing, may derive the carbon dioxide concentration based on a corrected third pump current obtained by correcting the third pump current taking into account a result of the third inner electrode deterioration determination processing, or may derive the carbon dioxide concentration by correcting a provisional carbon dioxide concentration based on the third pump current taking into account a result of the third inner electrode deterioration determination processing, and the control device, in the carbon dioxide concentration measurement processing, may perform a correction such that the greater the difference in the third inner electrode degradation determination processing, the more the absolute value of the third pump current or the provisional carbon dioxide concentration tends to be increased. In this way, since more appropriate correction can be performed according to the degree of degradation of the third inner electrode, it is possible to further suppress a decrease in the measurement accuracy of the carbon dioxide concentration due to degradation of the third inner electrode.
[7] In the above gas sensor (the gas sensor described in any one of [1] to [6]), the measurement gas may be exhaust gas of an internal combustion engine, and the control device, in the third inner electrode degradation determination processing, may be configured not to perform a determination of degradation of the third inner electrode when an operating state of the internal combustion engine differs between the time of measurement of the third voltage and the time of measurement of the third pump current. Here, when the operating state of the internal combustion engine differs, the carbon dioxide concentration in the measurement gas is also likely to differ. If the carbon dioxide concentration differs between the time of measurement of the third voltage and the time of measurement of the third pump current, the correspondence relationship between the third voltage and the third pump current changes due to factors other than degradation of the third inner electrode, and therefore the accuracy of determining the degradation of the third inner electrode may decrease. Accordingly, by not performing the determination of the degradation of the third inner electrode when the operating state of the internal combustion engine differs between the time of measurement of the third voltage and the time of measurement of the third pump current, it is possible to suppress a decrease in the accuracy of determining the degradation of the third inner electrode.
[8] In the above gas sensor (the gas sensor described in any one of [1] to [7]), the control device, in the third inner electrode degradation determination processing, may be configured not to perform a determination of degradation of the third inner electrode when an absolute value of the third voltage falls within a predetermined high-voltage region. Here, when the second inner electrode degrades, an ability of the second inner electrode to oxidize hydrogen decreases, so that a portion of the hydrogen that has reached the second chamber is not oxidized and reaches the third chamber. In this case, the absolute value of the third voltage becomes larger than when almost no hydrogen reaches the third chamber. The inventors have confirmed this through experiments and analysis. Accordingly, in a state where the second inner electrode is degraded, even if the third inner electrode is not degraded, the correspondence relationship between the third voltage and the third pump current changes, and the accuracy of determining the degradation of the third inner electrode may decrease. Therefore, by not performing the determination of the degradation of the third inner electrode when the absolute value of the third voltage falls within a predetermined high-voltage region, it is possible to suppress a decrease in the accuracy of determining the degradation of the third inner electrode.
[9] In the above gas sensor (the gas sensor described in any one of [1] to [7]), the control device may perform a second inner electrode degradation determination processing for determining degradation of the second inner electrode based on whether or not an absolute value of the third voltage during execution of the first and second pump cell control processing and during stoppage of the third pump cell control processing falls within a predetermined high-voltage region, and when the control device determines in the second inner electrode degradation determination processing that the second inner electrode is degraded, the control device may be configured not to perform a determination of degradation of the third inner electrode in the third inner electrode degradation determination processing. As described above, since the absolute value of the third voltage increases when the second inner electrode degrades, it is possible to determine the degradation of the second inner electrode based on whether or not the absolute value of the third voltage falls within the predetermined high-voltage region. That is, in this gas sensor, not only the degradation of the third inner electrode but also the degradation of the second inner electrode can be determined. Furthermore, since the control device does not perform the determination of the degradation of the third inner electrode when the control device determines that the second inner electrode is degraded, it is possible to suppress a decrease in the accuracy of determining the degradation of the third inner electrode.
[10] In the above gas sensor (the gas sensor described in [9]), the measurement gas may be exhaust gas of an internal combustion engine, and the control device may perform the second inner electrode degradation determination processing during fuel cut of the internal combustion engine or during stoppage thereof. Since the exhaust gas during fuel cut of the internal combustion engine or during stoppage thereof has a lower carbon dioxide concentration compared to the exhaust gas during operation other than fuel cut of the internal combustion engine, the influence of carbon monoxide reaching the third chamber becomes small, and the influence of hydrogen becomes dominant, regarding the magnitude of the absolute value of the third voltage. Therefore, during fuel cut or stoppage of the internal combustion engine, the timing is suitable for performing the second inner electrode degradation determination processing.
[11] In the above gas sensor (the gas sensor described in any one of [1] to [10]), the control device may measure a water concentration in the measurement gas based on the second pump current flowing through the second pump cell by the second pump cell control processing. In this way, this gas sensor can measure not only the carbon dioxide concentration but also the water concentration.

### BRIEF DESCRIPTION OF THE DRAWINGS

FIG. 1 is a schematic cross-sectional view schematically showing an example of a configuration of a gas sensor 100.

FIG. 2 is a block diagram showing the electrical connections between a control device 95 and respective cells and other components.

FIG. 3 is a flowchart showing one example of a first processing routine.

FIG. 4 is a graph showing a change in voltage V2 depending on whether or not the first measurement electrode 51 is degraded.

FIG. 5 is a graph showing relationships between a carbon monoxide concentration and a hydrogen concentration and the voltage V2.

FIG. 6 is a flowchart showing one example of a second processing routine.

FIG. 7 is an explanatory diagram showing one example of a table of correction patterns.

FIG. 8 is a graph showing a relationship between a carbon dioxide concentration and the voltage V2.

FIG. 9 is a graph showing a relationship between the carbon dioxide concentration and a pump current Ip2.

FIG. 10 is a graph showing a relationship between the voltage V2 and the pump current Ip2 corresponding to the same carbon dioxide concentration.

FIG. 11 is a graph showing a relationship between a measured value of the pump current Ip2 and a ratio Ip2c/Ip2.

FIG. 12 is a flowchart showing one example of a concentration measurement processing routine.

FIG. 13 is a schematic cross-sectional view of a sensor element 201 according to a modification.

### DETAILED DESCRIPTION OF THE INVENTION

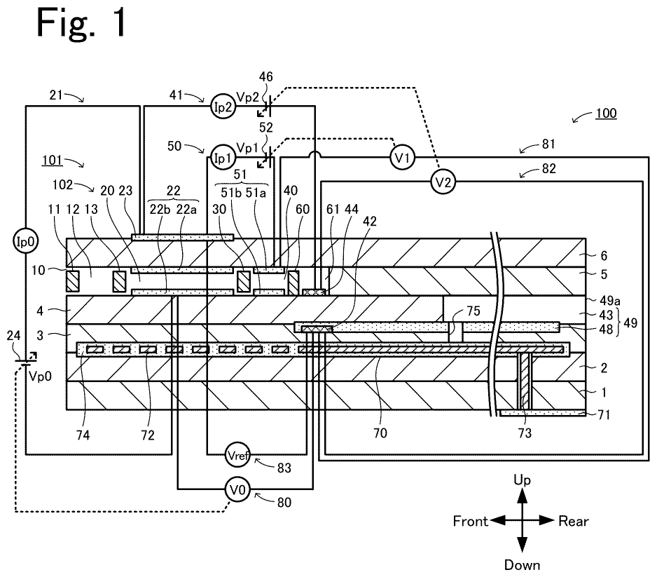

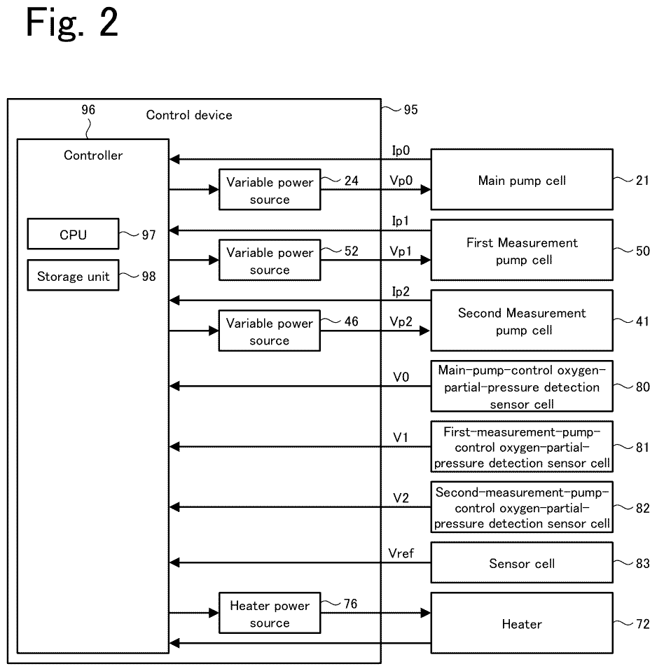

Next, embodiments of the present invention will be described with reference to the drawings. FIG. 1 is a schematic cross-sectional view schematically showing an example of a configuration of a gas sensor 100 according to an embodiment of the present invention. FIG. 2 is a block diagram showing the electrical connections between a control device 95, respective cells and a heater 72. The gas sensor 100 is installed in a pipe, such as an exhaust pipe of an internal combustion engine. The gas sensor 100 measures a concentration of a specific gas in a measurement gas, using exhaust gas from an internal combustion engine as the measurement gas. In the present embodiment, the gas sensor 100 is configured to detect the water concentration and the carbon dioxide concentration as the concentrations of the specific gases. The gas sensor 100 includes: a sensor element 101 with an elongated rectangular parallelepiped element body 102; cells 21, 41, 50, and 80 to 83 within the sensor element 101; a heater section 70 provided inside the sensor element 101; and a control device 95, which includes variable power sources 24, 46, and 52, and a heater power source 76, and controls the overall operation of the gas sensor 100. Note that the longitudinal direction (left-right direction in FIG. 1) of the sensor element 101 is defined as the front-rear direction, the thickness direction (up-down direction in FIG. 1) of the sensor element 101 as the up-down direction, and the width direction (perpendicular to both front-rear direction and up-down direction) of the sensor element 101 as the left-right direction.

The element body 102 is a laminated body in which six layers are stacked in the following order from the bottom in the drawing: a first substrate layer 1, a second substrate layer 2, a third substrate layer 3, a first solid electrolyte layer 4, a spacer layer 5, and a second solid electrolyte layer 6. Each of these layers is composed of an oxygen-ion-conductive solid electrolyte layer, such as zirconia (ZrO₂) or the like. The solid electrolytes forming these six layers are dense and hermetically sealed. The element body 102 is manufactured, for example, by performing predetermined processing and printing of circuit patterns on ceramic green sheets corresponding to the respective layers, laminating the sheets, and then firing the laminated sheets to integrate them into a unified structure.

On the front end side of the sensor element 101 (element body 102), between the lower surface of the second solid electrolyte layer 6 and the upper surface of the first solid electrolyte layer 4, the following components are formed adjacently and connected in sequence: a gas inlet 10; a first diffusion rate-limiting section 11; a buffer space 12; a second diffusion rate-limiting section 13; a first internal cavity 20; a third diffusion rate-limiting section 30; a second internal cavity 40; a fourth diffusion rate-limiting section 60; and a third internal cavity 61.

The gas inlet 10, buffer space 12, first internal cavity 20, second internal cavity 40, and third internal cavity 61 are internal spaces within the sensor element 101, formed by hollowing out portions of the spacer layer 5. These spaces are bounded at the top by the lower surface of the second solid electrolyte layer 6, at the bottom by the upper surface of the first solid electrolyte layer 4, and on the sides by the side surfaces of the spacer layer 5.

The first diffusion rate-limiting section 11, the second diffusion rate-limiting section 13, and the third diffusion rate-limiting section 30 are each provided as two horizontally elongated slits, with openings oriented along the longitudinal direction perpendicular to the plane of the drawing. The fourth diffusion rate-limiting section 60 is provided as a single horizontally elongated slit, with openings oriented along the longitudinal direction perpendicular to the plane of the drawing, formed as a gap with the lower surface of the second solid electrolyte layer 6. The area extending from the gas inlet 10 to the third internal cavity 61 is also referred to as the measurement gas flow path.

The sensor element 101 (element body 102) includes a reference gas introduction portion 49, which introduces a reference gas from outside of the sensor element 101 to a reference electrode 42 when measuring the concentrations of the specific gases. The reference gas introduction portion 49 comprises a reference gas introduction space 43 and a reference gas introduction layer 48. The reference gas introduction space 43 is an inward space formed from the rear end surface of the sensor element 101. The reference gas introduction space 43 is located between the upper surface of the third substrate layer 3 and the lower surface of the spacer layer 5, and is laterally defined by the side surfaces of the first solid electrolyte layer 4. The reference gas introduction space 43 opens to the rear end surface of the sensor element 101, with this opening serving as an inlet portion 49a of the reference gas introduction portion 49. The reference gas is introduced into the reference gas introduction space 43 through the inlet portion 49a. The reference gas introduction portion 49 introduces the reference gas, which has entered through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina or the like. A portion of the upper surface of the reference gas introduction layer 48 is exposed within the reference gas introduction space 43. The reference gas introduction layer 48 is formed so as to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4, and as described above, the reference gas introduction layer 48, which is connected to the reference gas introduction space 43, is provided around the reference electrode 42. Furthermore, as will be explained later, the reference electrode 42 enables the measurement of the oxygen concentration (oxygen partial pressure) in the first internal cavity 20, the second internal cavity 40, and the third internal cavity 61. The reference electrode 42 is formed as a porous cermet electrode (for example, a cermet electrode of Pt and ZrO₂).

In the measurement gas flow path, the gas inlet 10 is a portion that is open to the external space, allowing the measurement gas to be drawn into the sensor element 101 from the external space. The first diffusion rate-limiting section 11 is a part that imparts a predetermined diffusion resistance to the measurement gas introduced through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. When the measurement gas is introduced from outside the sensor element 101 into the first internal cavity 20, the measurement gas that is abruptly drawn into the sensor element 101 through the gas inlet 10 due to pressure fluctuations in the external space (such as exhaust pulsations in the case where the measurement gas is automobile exhaust gas) is not directly introduced into the first internal cavity 20. Instead, after the pressure fluctuations of the measurement gas are attenuated through the first diffusion rate-limiting section 11, the buffer space 12, and the second diffusion rate-limiting section 13, the measurement gas is introduced into the first internal cavity 20. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of a main pump cell 21.

The main pump cell 21 is an electrochemical pump cell, which is constituted by an inner pump electrode 22 with a ceiling electrode portion 22a provided on nearly the entire lower surface of the second solid electrolyte layer 6 facing the first internal cavity 20, an outer pump electrode 23, which is provided in a manner exposed to the outside of the sensor element 101 in a region of the upper surface of the second solid electrolyte layer 6 corresponding to the ceiling electrode portion 22a, and the second solid electrolyte layer 6, the spacer layer 5, and the first solid electrolyte layer 4, which form the current path between these electrodes.

The inner pump electrode 22 is formed so as to extend across the upper and lower solid electrolyte layers, (namely the second solid electrolyte layer 6 and the first solid electrolyte layer 4,) and the spacer layer 5 that provides sidewalls, which together define the first internal cavity 20. Specifically, the ceiling electrode portion 22a is formed on the lower surface of the second solid electrolyte layer 6, which constitutes the ceiling surface of the first internal cavity 20, and a bottom electrode portion 22b is formed on the upper surface of the first solid electrolyte layer 4, which constitutes the bottom surface of the first internal cavity 20. Further, in order to connect the ceiling electrode portion 22a and the bottom electrode portion 22b, side electrode portions (not shown) are formed on the side wall surfaces (inner surfaces) of the spacer layer 5, which constitute both sidewall portions of the first internal cavity 20. The inner pump electrode 22 is disposed in a tunnel-like structure at the region where the side electrode portion is provided.

In the main pump cell 21, a desired voltage Vp0 is applied between the inner pump electrode 22 and the outer pump electrode 23, whereby a pump current Ip0 is caused to flow in a positive direction or a negative direction between the inner pump electrode 22 and the outer pump electrode 23. Thus, the oxygen in the first internal cavity 20 can be pumped out to the external space, or the oxygen in the external space can be pumped into the first internal cavity 20.

Further, in order to detect the oxygen concentration (oxygen partial pressure) in the atmosphere within the first internal cavity 20, an electrochemical sensor cell, that is, a main-pump-control oxygen-partial-pressure detection sensor cell 80, is constituted by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, by feedback-controlling the voltage Vp0 of the variable power source 24 such that the voltage V0 reaches a target value, the pump current Ip0 is controlled, thereby adjusting the oxygen concentration in the first internal cavity 20.

The third diffusion rate-limiting section 30 is a part that imparts a predetermined diffusion resistance to the measurement gas, whose oxygen concentration (oxygen partial pressure) is controlled by the operation of the main pump cell 21 in the first internal cavity 20, and guides the measurement gas into the second internal cavity 40.

The second internal cavity 40 is provided as a space in which the oxygen partial pressure is adjusted by the first measurement pump cell 50 for the measurement gas introduced through the third diffusion rate-limiting section 30, to carry out processing for measuring the water concentration in the measurement gas.

The first measurement pump cell 50 is an electrochemical pump cell, which is constituted by a first measurement electrode 51 with a ceiling electrode portion 51a provided on nearly the entire lower surface of the second solid electrolyte layer 6 facing the second internal cavity 40, the outer pump electrode 23 (not limited to the outer pump electrode 23, but may be any suitable electrode disposed on an outer surface of the sensor element 101), the second solid electrolyte layer 6, the spacer layer 5, and the first solid electrolyte layer 4.

The first measurement electrode 51 is disposed within the second internal cavity 40 in a tunnel-like structure similar to that of the inner pump electrode 22 disposed in the first internal cavity 20 described above. Specifically, the ceiling electrode portion 51a is formed on the second solid electrolyte layer 6, which constitutes the ceiling surface of the second internal cavity 40, and a bottom electrode portion 51b is formed on the first solid electrolyte layer 4, which constitutes the bottom surface of the second internal cavity 40. Further, side electrode portions (not shown), which connect the ceiling electrode portion 51a and the bottom electrode portion 51b, are formed on the inner side surfaces of the spacer layer 5, which constitute both sidewall portions of the second internal cavity 40. Thus, the first measurement electrode 51 is formed in a tunnel-like structure.

In the first measurement pump cell 50, a desired voltage Vp1 is applied between the first measurement electrode 51 and the outer pump electrode 23. Thus, the oxygen in the atmosphere within the second internal cavity 40 can be pumped out to the external space, or the oxygen can be pumped into the second internal cavity 40 from the external space.

Further, in order to control the oxygen partial pressure in the atmosphere within the second internal cavity 40, an electrochemical sensor cell, that is, an first-measurement-pump-control oxygen-partial-pressure detection sensor cell 81, is constituted by the first measurement electrode 51, the reference electrode 42, the second solid electrolyte layer 6, the spacer layer 5, the first solid electrolyte layer 4, and the third substrate layer 3.

The first measurement pump cell 50 performs pumping via the variable power source 52, which is voltage-controlled based on an electromotive force (voltage V1) detected by the first-measurement-pump-control oxygen-partial-pressure detection sensor cell 81. As a result, the oxygen partial pressure in the atmosphere of the second internal cavity 40 is adjusted by the pump current Ip1 flowing through the first measurement pump cell 50.

The fourth diffusion rate-limiting section 60 is a part that imparts a predetermined diffusion resistance to the measurement gas, whose oxygen concentration (oxygen partial pressure) is controlled by the operation of the first measurement pump cell 50 in the second internal cavity 40, and guides the measurement gas into the third internal cavity 61.

The third internal cavity 61 is provided as a space in which the oxygen partial pressure is adjusted by the second measurement pump cell 41 for the measurement gas introduced through the fourth diffusion rate-limiting section 60, to carry out processing for measuring the carbon dioxide concentration in the measurement gas.

The second measurement pump cell 41 is an electrochemical pump cell, which is constituted by a second measurement electrode 44 provided on the upper surface of the first solid electrolyte layer 4 facing the third internal cavity 61, the outer pump electrode 23 (not limited to the outer pump electrode 23, but may be any suitable electrode disposed on an outer surface of the sensor element 101), the second solid electrolyte layer 6, the spacer layer 5, and the first solid electrolyte layer 4.

In the second measurement pump cell 41, a desired voltage Vp2 is applied between the second measurement electrode 44 and the outer pump electrode 23. Thus, the oxygen in the atmosphere within the third internal cavity 61 can be pumped out to the external space, or the oxygen can be pumped into the second internal cavity 40 from the external space.

Further, in order to detect the oxygen partial pressure around the second measurement electrode 44, an electrochemical sensor cell, that is, a second-measurement-pump-control oxygen-partial-pressure detection sensor cell 82, is formed by the first solid electrolyte layer 4, the third substrate layer 3, the second measurement electrode 44, and the reference electrode 42.

The variable power source 46 is controlled based on the electromotive force (voltage V2) detected by the second-measurement-pump-control oxygen-partial-pressure detection sensor cell 82, and the voltage Vp2 of the variable power source 46 is applied to the second measurement pump cell 41. As a result, the oxygen partial pressure in the atmosphere of the third internal cavity 61 is adjusted by the pump current Ip2 flowing through the second measurement pump cell 41.

Furthermore, an electrochemical sensor cell 83 is formed from the second solid electrolyte layer 6, the spacer layer 5, the first solid electrolyte layer 4, the third substrate layer 3, the outer pump electrode 23, and the reference electrode 42, and the oxygen partial pressure in the measurement gas outside the sensor can be detected based on the electromotive force (voltage Vref) obtained by this sensor cell 83.

Each of the electrodes 22, 23, 42, 44, and 51 will be described. The inner pump electrode 22, the first measurement electrode 51, and the second measurement electrode 44 each contain a first type of noble metal with catalytic activity. Examples of the first type of noble metal include, but are not limited to, at least one selected from Pt, Rh, Ir, Ru, and Pd. The outer pump electrode 23 and the reference electrode 42 also contain the first type of noble metal. It is preferable that the first measurement electrode 51 further contain a second type of noble metal for suppressing the catalytic activity of the first type of noble metal with respect to carbon monoxide. By containing the second type of noble metal, the oxidation capability of the first measurement electrode 51 with respect to carbon monoxide is reduced. An example of the second type of noble metal is Au. The inner pump electrode 22 and the second measurement electrode 44 do not contain the second type of noble metal. Preferably, the outer pump electrode 23 and the reference electrode 42 also do not contain the second type of noble metal. Each of the electrodes 22, 23, 42, 44, and 51 is preferably a cermet containing a noble metal and a solid electrolyte having oxygen ion conductivity (e.g. ZrO₂). Each of the electrodes 22, 23, 42, 44, and 51 is also preferably a porous body. In the present embodiment, the first measurement electrode 51 is a porous cermet electrode composed of Pt containing 1% Au and ZrO₂. Additionally, the inner pump electrode 22, the outer pump electrode 23, the reference electrode 42, and the second measurement electrode 44 are each porous cermet electrodes composed of Pt and ZrO₂.

The sensor element 101 includes a heater section 70 that performs temperature regulation by heating and maintaining the temperature of the sensor element 101, in order to enhance the oxygen-ion-conductivity of the solid electrolyte. The heater section 70 includes a heater connector electrode 71, a heater 72, a through hole 73, a heater insulating layer 74, and a pressure relief hole 75.

The heater connector electrode 71 is an electrode formed in such a manner as to be in contact with the lower surface of the first substrate layer 1. By connecting the heater connector electrode 71 to a heater power source 76 (see FIG. 2), power can be supplied from the heater power source 76 to the heater section 70.

The heater 72 is an electrical resistor formed in such a manner as to be sandwiched between the second substrate layer 2 and the third substrate layer 3 from above and below. The heater 72 is connected to the heater connector electrode 71 via the through hole 73, and generates heat when power is supplied from the heater power source 76 through the heater connector electrode 71, thereby heating and maintaining the temperature of the solid electrolyte forming the sensor element 101.

The heater 72 is also embedded across the entire region from the first internal cavity 20 to the third internal cavity 61, making it possible to adjust the temperature of the entire sensor element 101 to a level at which the solid electrolyte is activated.

The heater insulating layer 74 is an insulating layer formed of an insulator such as alumina and provided on the upper and lower surfaces of the heater 72. The heater insulating layer 74 is formed for the purpose of providing electrical insulation between the second substrate layer 2 and the heater 72, as well as between the third substrate layer 3 and the heater 72.

The pressure relief hole 75 is a portion that penetrates through the third substrate layer 3 and the reference gas introduction layer 48, and is formed so as to communicate with the reference gas introduction space 43. The pressure relief hole 75 is formed for the purpose of relieving an increase in internal pressure caused by a rise in temperature within the heater insulating layer 74.

As shown in FIG. 2, the control device 95 includes the variable power sources 24, 46, and 52 described above, the heater power source 76 also described above, and a control unit 96. The control unit 96 is a microprocessor including a CPU 97 and a storage unit 98. The storage unit 98 is a rewritable non-volatile memory, and is capable of storing, for example, various programs and various kinds of data. The control unit 96 inputs the voltage V0 from the main-pump-control oxygen-partial-pressure detection sensor cell 80, the voltage V1 from the first-measurement-pump-control oxygen-partial-pressure detection sensor cell 81, the voltage V2 from the second-measurement-pump-control oxygen-partial-pressure detection sensor cell 82, the voltage Vref from the sensor cell 83, the pump current Ip0 flowing through the main pump cell 21, the pump current Ip1 flowing through the first measurement pump cell 50, and the pump current Ip2 flowing through the second measurement pump cell 41. In addition, the control unit 96 controls the voltages Vp0, Vp1, and Vp2 output from the variable power sources 24, 52, and 46, respectively, by outputting control signals to the variable power sources 24, 52, and 46. Through this control, the main pump cell 21, the first measurement pump cell 50, and the second measurement pump cell 41 are controlled. The control unit 96 also controls the power supplied from the heater power source 76 to the heater 72 by outputting a control signal to the heater power source 76. The storage unit 98 also stores target values V0*, V1*, and V2*, which will be described later. The CPU 97 of the control unit 96 performs control of the respective cells 21, 50, and 41 with reference to the target values V0*, V1*, and V2*.

The control unit 96 performs a main pump control processing (an example of the first pump cell control processing), which controls the main pump cell 21 to pump out oxygen from around the inner pump electrode 22 to around the outer pump electrode 23. Specifically, the control unit 96 feedback-controls the voltage Vp0 of the variable power source 24 so that the voltage V0 reaches a target value V0*, thereby controlling the main pump cell 21. The target value V0* is set as a value such that the oxygen concentration in the first internal cavity 20 reaches a predetermined low concentration that is sufficiently low to substantially reduce all of the water and carbon dioxide in the measurement gas. By performing this main pump control processing, in the first internal cavity 20, water in the measurement gas is reduced to generate hydrogen and oxygen, and carbon dioxide in the measurement gas is reduced to generate carbon monoxide and oxygen. The generated oxygen is pumped out from around the inner pump electrode 22 to around the outer pump electrode 23 by the pump current Ip0 flowing through the main pump cell 21.

The control unit 96 performs a first measurement pump control processing (an example of the second pump cell control processing), which controls the first measurement pump cell 50 to pump into oxygen from around the outer pump electrode 23 to around the first measurement electrode 51. Specifically, the control unit 96 feedback-controls the voltage Vp1 of the variable power source 52 so that the voltage V1 reaches the target value V1*, thereby controlling the first measurement pump cell 50. The target value V1* is set as a value such that the oxygen concentration in the second internal cavity 40 reaches a predetermined concentration sufficient to substantially oxidize all of hydrogen in the second internal cavity 40. By performing this first measurement pump control processing, in the second internal cavity 40, hydrogen generated by the reduction of water in the first internal cavity 20 is oxidized to generate water again. At this time, the pump current Ip1 flowing through the first measurement pump cell 50 correlates with the amount of oxygen pumped into the second internal cavity 40 to oxidize the hydrogen in the second internal cavity 40, and hence correlates with the amount of water in the measurement gas in the first internal cavity 20, which is the source of the hydrogen in the second internal cavity 40. Therefore, the pump current Ip1 correlates with the water concentration in the measurement gas and the water concentration in the measurement gas can be measured based on the pump current Ip1.

The control unit 96 performs a second measurement pump control processing (an example of the third pump cell control processing), which controls the second measurement pump cell 41 to pump into oxygen from around the outer pump electrode 23 to around the second measurement electrode 44. Specifically, the control unit 96 feedback-controls the voltage Vp2 of the variable power source 46 so that the voltage V2 reaches the target value V2*, thereby controlling the second measurement pump cell 41. The target value V2* is set as a value such that the oxygen concentration in the third internal cavity 61 reaches a predetermined concentration sufficient to substantially oxidize all of carbon monoxide in the third internal cavity 61. By performing this second measurement pump control processing, in the third internal cavity 61, carbon monoxide generated by the reduction of carbon dioxide in the first internal cavity 20 is oxidized to generate carbon dioxide again. At this time, the pump current Ip2 flowing through the second measurement pump cell 41 correlates with the amount of oxygen pumped into the third internal cavity 61 to oxidize the carbon monoxide in the third internal cavity 61, and hence correlates with the amount of carbon dioxide in the measurement gas in the first internal cavity 20, which is the source of the carbon monoxide in the third internal cavity 61. Therefore, the pump current Ip2 correlates with the carbon dioxide concentration in the measurement gas and the carbon dioxide concentration in the measurement gas can be measured based on the pump current Ip2.

Both hydrogen and carbon monoxide generated in the first internal cavity 20 reach the second internal cavity 40. However, hydrogen has a faster gas diffusion rate than carbon monoxide and hydrogen binds with oxygen more readily than carbon monoxide does. Therefore, in the second internal cavity 40, hydrogen can be selectively oxidized, among hydrogen and carbon monoxide, by the first measurement pump control processing. Additionally, since hydrogen rarely reaches the third internal cavity 61 downstream of the second internal cavity 40, the second measurement pump control processing can oxidize carbon monoxide. Further, in this embodiment, as described above, the first measurement electrode 51 has its oxidation capability with respect to carbon monoxide reduced by containing the second type of noble metal. Therefore, in a vicinity of the first measurement electrode 51, that is, in the second internal cavity 40, hydrogen can be more selectively oxidized than carbon monoxide by the first measurement pump control processing.

The control unit 96 performs heater control processing by outputting a control signal to the heater power source 76 such that the temperature of the heater 72 reaches to a target temperature (e.g. 800° C.). Here, the target temperature of the heater 72 is defined as a temperature obtained by adding a margin to the temperature at which the above-described solid electrolyte is activated. The temperature of the heater 72 can be expressed as a linear function of the resistance value of the heater 72. In the heater control processing, the control unit 96 calculates the resistance value of the heater 72, which is a value can be regarded as the temperature of the heater 72 (a value that can be converted into temperature), and feedback-controls the heater power source 76 such that the calculated resistance value reaches to a target resistance value (a value corresponding to the target temperature). The control unit 96, for example, can acquire the voltage of the heater 72 and the current flowing through the heater 72, then calculate the resistance of the heater 72 based on the acquired voltage and current. The control unit 96 may use a three-wire or four-wire method to calculate the resistance, for example. The heater power source 76 adjusts the power supplied to the heater 72 by changing the voltage applied to the heater 72 based on a control signal from the control unit 96 when energizing the heater 72.

In addition, the control device 95, which includes the variable power sources 24, 46, 52, and the heater power source 76 shown in FIG. 2, is actually connected to the electrodes inside the sensor element 101 through unillustrated lead wires formed within the sensor element 101 and unillustrated connector electrodes formed at the rear end of the sensor element 101 (only the heater connector electrode 71 shown in FIG. 1).

When the gas sensor 100 configured as described above is used, the CPU 97 of the control unit 96 first performs the heater control processing described above to control the heater 72 such that its temperature reaches the target temperature. When the temperature of the heater 72 reaches the target temperature (or near the target temperature), the CPU 97 starts the control of each pump cell 21, 41, and 50 (main pump control processing, first measurement pump control processing, and second measurement pump control processing) described above, and starts acquiring each of the voltages V0, V1, V2, and Vref from each of the sensor cells 80 to 83 described above. While continuously performing these processes, the control unit 96 performs a degradation determination processing for the first measurement electrode 51, a degradation determination processing for the second measurement electrode 44, and a concentration measurement processing, which will be described later. In the present embodiment, the period from the start to the end of the heater control processing is defined as one use of the gas sensor 100. For example, the control unit 96 starts the heater control processing upon receiving a command from an engine ECU (not shown) at the start of operation of the internal combustion engine, and ends the heater control processing upon receiving a command from the engine ECU at the stop of operation of the internal combustion engine.

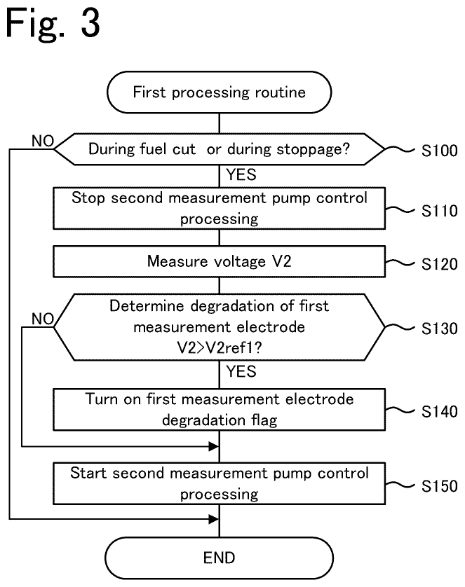

Next, an example of the degradation determination processing for the first measurement electrode 51 will be described. FIG. 3 is a flowchart showing one example of a first processing routine including the degradation determination processing for the first measurement electrode 51. This routine is stored, for example, in the storage unit 98 of the control unit 96 and is repeatedly executed by the CPU 97.

When the first processing routine of FIG. 3 is executed, the CPU 97 first determines whether or not the internal combustion engine is in a fuel cut state or stopped (step S100). For example, CPU 97 acquires information that can identify whether the internal combustion engine is in a fuel cut state or not, and information that can identify whether the internal combustion engine is stopped or not from the engine ECU, and performs the determination in step S100 based on the acquired information. Alternatively, the CPU 97 may detect the oxygen concentration in the measurement gas around the sensor element 101 based on the voltage Vref of the sensor cell 83 described above, and perform the determination of step S100 based on whether or not the detected oxygen concentration falls within a high concentration region that can be regarded as a fuel cut state or stopped state. If the CPU 97 determines in step S100 that the internal combustion engine is neither in a fuel cut state nor stopped, this routine is terminated.

If the CPU 97 determines in step S100 that the internal combustion engine is in a fuel cut state or stopped, the CPU 97 stops the second measurement pump control processing (step S110) and measures the voltage V2 in that state (step S120). That is, the CPU 97 measures the voltage V2 while the main pump control processing and the first measurement pump control processing are being executed and the second measurement pump control processing is stopped. Then, the CPU 97 performs the degradation determination processing for the first measurement electrode 51 based on the measured voltage V2 (step S130). The CPU 97 determines whether or not the first measurement electrode 51 is degraded based on whether or not the absolute value of the measured voltage V2 falls within a predetermined high-voltage region. For example, the CPU 97 determines that the first measurement electrode 51 is degraded when the absolute value of the measured voltage V2 is greater than a predetermined threshold value V2ref1, and determines that the first measurement electrode 51 is not degraded when the absolute value is equal to or less than the threshold value V2ref1. When the absolute value of the voltage V2 is greater than the threshold value V2ref1 in step S130, the CPU 97 turns on a first measurement electrode degradation flag (step S140), starts (restarts) the second measurement pump control processing (step S150), and terminates this routine. When the absolute value of the voltage V2 is equal to or less than the threshold value V2ref1 in step S130, the CPU 97 terminates this routine by performing step S150 without turning on the first measurement electrode degradation flag. It is preferable that when the CPU 97 determines in step S130 that the first measurement electrode 51 is degraded, CPU 97 notifies an abnormality of the gas sensor 100 to another device such as the engine ECU or to the user such as the driver. Further, when the degradation of the first measurement electrode 51 is resolved, for example, such as after the sensor element 101 with the degraded first measurement electrode 51 has been replaced, the CPU 97 turns off the first measurement electrode degradation flag based on an operation from an operator.

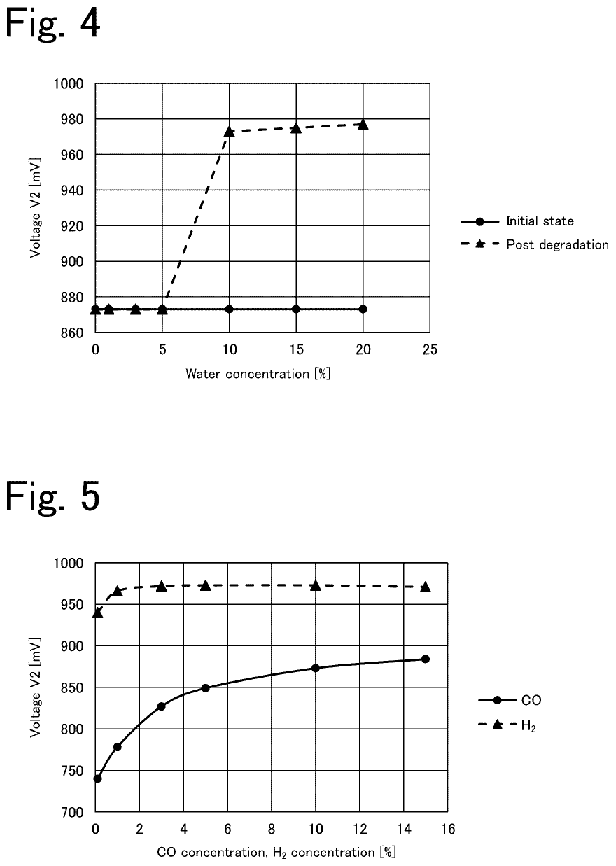

FIG. 4 is a graph showing a change in the voltage V2 depending on whether or not the first measurement electrode 51 is degraded. FIG. 5 is a graph showing relationships between a carbon monoxide concentration and a hydrogen concentration and the voltage V2. The inventors conducted the following experiments on the gas sensor 100 and obtained the graphs of FIG. 4 and FIG. 5. First, the gas sensor 100 provided with the sensor element 101 in an unused (initial) state and the gas sensor 100 provided with the sensor element 101 after degradation of the first measurement electrode 51 were prepared. The sensor element 101, whose first measurement electrode 51 had been degraded by performing the heater control processing and the first measurement pump control processing for 1000 hours while exposing the tip end of the sensor element 101 to the exhaust gas of the internal combustion engine, was used as the degraded sensor element 101.

Next, for each of the sensor element 101 in the initial state and the sensor element 101 after degradation, the relationship between the water concentration in the measurement gas and the voltage V2 was examined, and the graph of FIG. 4 was obtained. Specifically, the gas sensor 100 provided with the sensor element 101 in the initial state was attached to a pipe such that the tip portion of the sensor element 101 protruded into the inside of the pipe. Then, a model gas was supplied to the pipe as the measurement gas, while the control unit 96 executed the heater control processing, the main pump control processing, the first measurement pump control processing, and the second measurement pump control processing. As the model gas, a gas was used in which nitrogen was the base gas, the carbon dioxide concentration was set to 10%, and the water concentration was gradually varied. At predetermined intervals, steps S110 and S120 of FIG. 3 were executed, that is, the voltage V2 was measured while the main pump control processing and the first measurement pump control processing were executed and the second measurement pump control processing was stopped, and a plurality of data associating the measured voltage V2 with the water concentration of the model gas at that time were obtained. Similarly, a plurality of data associating the voltage V2 with the water concentration were obtained for the sensor element 101 after degradation in the same manner. The graph obtained by plotting these data is shown in FIG. 4.

Further, for the sensor element 101 in the initial state, the relationship between the carbon monoxide concentration and the hydrogen concentration and the voltage V2 was examined, and the graph of FIG. 5 was obtained. Specifically, the gas sensor 100 was attached to the pipe in the same manner as the experiment described above, and the voltage V2 was measured while supplying a model gas as the measurement gas. However, in this case, the control unit 96 executed the heater control processing, and did not execute the main pump control processing, the first measurement pump control processing, or the second measurement pump control processing. As the model gas, a gas containing carbon monoxide with nitrogen as the base gas was used. While gradually changing the carbon monoxide concentration of the model gas, the voltage V2 was measured, and a plurality of data associating the voltage V2 with the carbon monoxide concentration were obtained. Similarly, a model gas containing hydrogen with nitrogen as the base gas was used, and while gradually changing the hydrogen concentration of the model gas, the voltage V2 was measured, and a plurality of data associating the voltage V2 with the hydrogen concentration were obtained. The graph obtained by plotting these data is shown in FIG. 5.

As can be seen from FIG. 4, in the sensor element 101 in the initial state, the voltage V2 was almost constant (about 870 mV) regardless of the water concentration, and this value was almost equal to the value of the voltage V2 when the carbon monoxide concentration was 10% in FIG. 5. On the other hand, in the sensor element 101 after degradation, when the water concentration was 5% or less, the voltage V2 was the same value as the voltage V2 of the sensor element 101 in the initial state, but when the water concentration was 10% or more, the value of the voltage V2 was higher than that of the sensor element 101 in the initial state. In addition, the value of the voltage V2 of the sensor element 101 after degradation at that time was almost equal to the value of the voltage V2 when the hydrogen concentration was 10% or more in FIG. 5 (about 970 mV). This is considered to be because, when the first measurement electrode 51 degrades, the ability of the first measurement electrode 51 to oxidize hydrogen decreases, and even when the first measurement pump control processing is executed, at least a part of the hydrogen reaching the second internal cavity 40 is not oxidized and reaches the second measurement electrode 44 in the third internal cavity 61. By using this, in step S130 of FIG. 3 described above, whether or not the first measurement electrode 51 is degraded is determined based on whether or not the voltage V2 falls within a predetermined high-voltage region. The predetermined high-voltage region can be set in advance by experiments or the like as a region such that the absolute value of the voltage V2 does not reach when hydrogen is almost absent around the second measurement electrode 44 even if carbon monoxide is present, but reaches when hydrogen is present around the second measurement electrode 44. In the present embodiment, the threshold value V2ref1 described above is set to 920 mV as a value that can distinguish between the voltage V2 when the carbon monoxide concentration is 15%, which is the highest in FIG. 5, and the voltage V2 when the hydrogen concentration is 0.1%, which is the lowest in FIG. 5, and the region exceeding this threshold is defined as the high-voltage region. Note that since the voltage V2 also varies depending on the structure of the sensor element 101 (for example, the positions of the second measurement electrode 44 and the reference electrode 42 relative to the heater 72 and so forth), the values of the voltage V2 shown in FIGS. 4 and 5 and the value of the threshold V2ref1 are merely examples.

Since, during execution of the second measurement pump control processing, the voltage V2 is controlled to become a target value V2*, the voltage V2 does not take a value corresponding to the hydrogen concentration or the carbon monoxide concentration around the second measurement electrode 44, and therefore the degradation determination of the first measurement electrode 51 based on the voltage V2 cannot be performed. For this reason, in step S120 the voltage V2 is measured with the second measurement pump control processing stopped in step S110. Further, if the main pump control processing is not performed, hydrogen is not generated from water in the measurement gas; and even if the main pump control processing is performed, when the first measurement pump control processing is not performed, hydrogen reaches the second measurement electrode 44 even if the first measurement electrode 51 is not degraded. Accordingly, the measurement of the voltage V2 in step S120 is performed while the main pump control processing and the first measurement pump control processing are being executed.

As described above, in FIG. 4, even with the sensor element 101 after degradation, when the water concentration is 5% or less, the voltage V2 is the same value as the voltage V2 of the sensor element 101 in the initial state. This is considered to be because, even for the first measurement electrode 51 after degradation, if the hydrogen reaching the second internal cavity 40 is small, all the hydrogen can be oxidized, and hydrogen does not reach the second measurement electrode 44. Therefore, if the first measurement electrode 51 further degrades, the voltage V2 will exceed the threshold V2ref1 even when the water concentration is 5% or less, and it is considered that degradation can be detected based on the voltage V2.

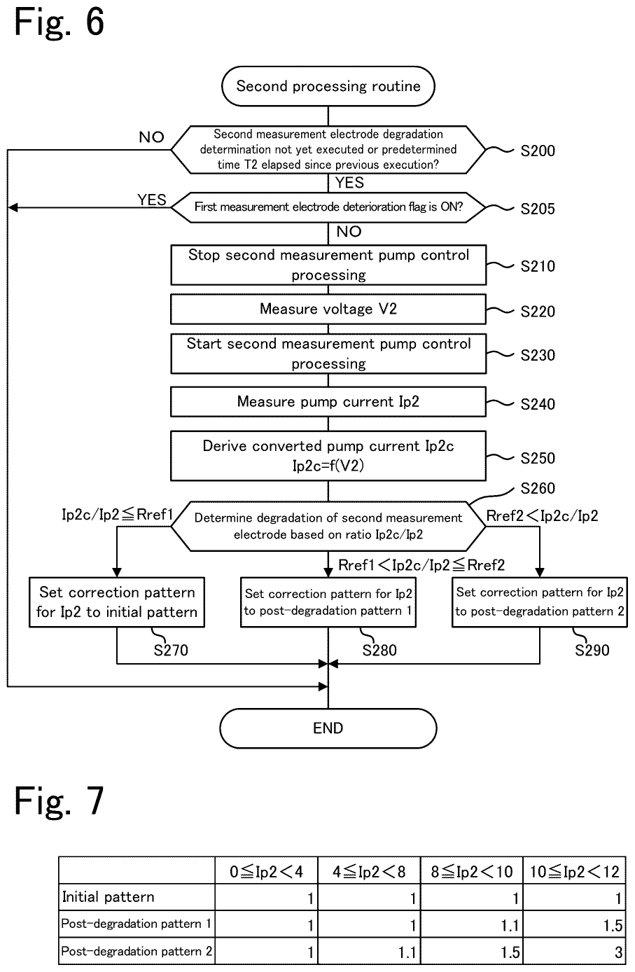

Subsequently, an example of the degradation determination processing for the second measurement electrode 44 will be described. FIG. 6 is a flowchart showing one example of a second processing routine including the degradation determination processing for the second measurement electrode 44. This routine is stored, for example, in the storage unit 98 of the control unit 96 and is repeatedly executed by the CPU 97.

When the second processing routine of FIG. 6 is executed, the CPU 97 first determines whether, in the current use of the gas sensor 100, the degradation determination processing for the second measurement electrode 44 (steps S250 and S260 described later) has not yet been executed or a predetermined time T2 has elapsed since the previous execution (step S200). The predetermined time T2 may be set, for example, to a time until the possibility arises that the second measurement electrode 44 will degrade from the initial state, or to a shorter time, and may be, for example, on the order of several seconds to several minutes, or on the order of several hours. If the CPU 97 determines that, in the current use of the gas sensor 100, the degradation determination processing for the second measurement electrode 44 has already been executed and the predetermined time T2 has not elapsed since the previous execution, this routine is terminated.

If the CPU 97 determines in step S200 that, in the current use of the gas sensor 100, the degradation determination processing for the second measurement electrode 44 has not been carried out, or determines that the predetermined time T2 has elapsed since the previous execution, the CPU 97 determines whether a first measurement electrode degradation flag is on (step S205). If the CPU 97 determines that the first measurement electrode degradation flag is on, this routine is terminated. That is, when it is determined in the first processing routine of FIG. 3 that the first measurement electrode 51 is degraded, the CPU 97 does not perform the determination of degradation of the second measurement electrode 44.

If the CPU 97 determines in step S205 that the first measurement electrode degradation flag is not on, the CPU 97 stops the second measurement pump control processing (step S210) and measures the voltage V2 in that state (step S220). That is, as in step S120 of FIG. 3 described above, the CPU 97 measures the voltage V2 while the main pump control processing and the first measurement pump control processing are being executed and the second measurement pump control processing is stopped. Subsequently, the CPU 97 starts (restarts) the second measurement pump control processing (step S230) and measures the pump current Ip2 (step S240). That is, the CPU 97 measures the pump current Ip2 while the main pump control processing, the first measurement pump control processing, and the second measurement pump control processing are being executed.

Then, based on the measured value of the voltage V2 and the measured value of the pump current Ip2, the CPU 97 performs the degradation determination processing for the second measurement electrode 44 (steps S250 and S260). Specifically, the CPU 97 derives a converted pump current Ip2c based on the measured value of the voltage V2 in step S220 (step S250), and determines the degradation of the second measurement electrode 44 based on a difference between the converted pump current Ip2c and the measured value of the pump current Ip2 in step S240 (step S260). In the present embodiment, as a value representing the difference between the converted pump current Ip2c and the pump current Ip2, a ratio Ip2c/Ip2 is used. As will be described later, the converted pump current Ip2c is a value corresponding to the pump current Ip2 in a case where the second measurement electrode 44 is not degraded. The ratio Ip2c/Ip2 normally becomes a value of 1 or more, and the larger the value of the ratio Ip2c/Ip2, that is, the larger the difference between the converted pump current Ip2c and the pump current Ip2, the more it indicates that the second measurement electrode 44 is degraded. Accordingly, in step S260, the CPU 97 determines the degradation of the second measurement electrode 44 by comparing the ratio Ip2c/Ip2 with thresholds Rref1 (>1) and Rref2 (>Rref1). The CPU 97 also determines a correction pattern for the pump current Ip2 when measuring the carbon dioxide concentration, in accordance with the determination result of degradation. Specifically, when the ratio Ip2c/Ip2 is equal to or less than the threshold Rref1, the CPU 97 determines that the second measurement electrode 44 is not degraded and sets the correction pattern for Ip2 to an initial pattern (step S270). When the ratio Ip2c/Ip2 is greater than the threshold Rref1 and equal to or less than the threshold Rref2, the CPU 97 determines that slight degradation has occurred in the second measurement electrode 44 and sets the correction pattern for Ip2 to a post-degradation pattern 1 (step S280). When the ratio Ip2c/Ip2 exceeds the threshold Rref2, the CPU 97 determines that significant degradation has occurred in the second measurement electrode 44 and sets the correction pattern for Ip2 to a post-degradation pattern 2 (step S290). After performing any one of steps S270 to S290 to set the correction pattern, the CPU 97 terminates this routine.

FIG. 7 is an explanatory diagram showing one example of a table of correction patterns stored in advance in the storage unit 98. In this table, for each of the initial pattern, the post-degradation pattern 1, and the post-degradation pattern 2, an absolute value of the pump current Ip2 before correction and a correction coefficient Ca to be multiplied by the absolute value of the pump current Ip2 before correction are associated with each other. The range of the pump current Ip2 before correction from 0 μA to 12 μA is divided into four regions, and, for each division, a value of the correction coefficient Ca is associated. In the initial pattern, the value 1 is associated as the correction coefficient Ca for any of the four regions. That is, when the correction pattern is set to the initial pattern, the pump current Ip2 is not corrected. In contrast, in both the post-degradation pattern 1 and the post-degradation pattern 2, the correction patterns are defined such that the correction coefficient Ca tends to become larger as the pump current Ip2 becomes larger (that is, such that the absolute value of the pump current Ip2 tends to be increased to a greater extent by correction). Further, when comparing the post-degradation pattern 1 with the post-degradation pattern 2, the correction patterns are defined such that, for the same value of the pump current Ip2, the correction coefficient Ca of the post-degradation pattern 2 tends to become larger (that is, the absolute value of the pump current Ip2 tends to be increased to a greater extent by correction).

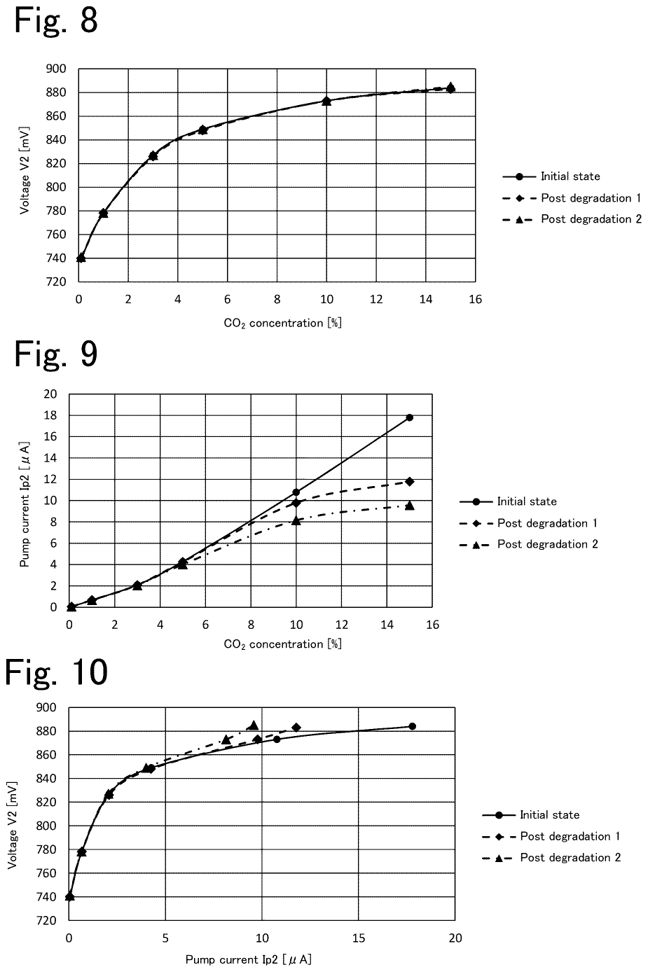

The reason for determining the degradation of the second measurement electrode 44 and setting the correction pattern based on the voltage V2 and the pump current Ip2, as described with reference to FIGS. 6 and 7, will be explained. FIG. 8 is a graph showing a relationship between the carbon dioxide concentration in the measurement gas and the voltage V2. FIG. 9 is a graph showing a relationship between the carbon dioxide concentration in the measurement gas and the pump current Ip2. FIG. 10 is a graph showing a relationship between the voltage V2 and the pump current Ip2 corresponding to the same carbon dioxide concentration. The inventors conducted the following experiments on the gas sensor 100 and obtained the graphs of FIGS. 8 to 10. First, the gas sensor 100 provided with the sensor element 101 in an unused (initial) state and the gas sensor 100 provided with the sensor element 101 after degradation of the second measurement electrode 44 were prepared. As the sensor element 101 after degradation, two types were prepared: a sensor element 101 of post-degradation 1 with a small degree of degradation and a sensor element 101 of post-degradation 1 with a large degree of degradation. As the sensor elements 101 of post-degradation 1 and 2, sensor elements 101 were used in which the second measurement electrode 44 was degraded by subjecting the tip side of the sensor element 101 to the exhaust gas of an internal combustion engine and executing the heater control processing and the second measurement pump control processing for a long time. The execution time of the heater control processing and the second measurement pump control processing was made longer for the sensor element 101 of post-degradation 2 than for the sensor element 101 of post-degradation 1.

Next, for each of the sensor element 101 in the initial state, the sensor element 101 of post-degradation 1, and the sensor element 101 of post-degradation 2, the correspondence relationship among the carbon dioxide concentration in the measurement gas, the voltage V2, and the pump current Ip2 was examined. Specifically, the gas sensor 100 provided with the sensor element 101 in the initial state was attached to a pipe such that the tip portion of the sensor element 101 protruded into the inside of the pipe. Then, while supplying a model gas to the pipe as the measurement gas, the control unit 96 executed the heater control processing, the main pump control processing, the first measurement pump control processing, and the second measurement pump control processing. As the model gas, a gas was used in which nitrogen was the base gas and the carbon dioxide concentration was gradually varied. At predetermined intervals, steps S210 to S240 of FIG. 6 were executed to obtain measured values of the voltage V2 and the pump current Ip2. During execution of steps S210 to S240, the carbon dioxide concentration of the model gas was kept from changing. Thus, a plurality of data associating the carbon dioxide concentration with the voltage V2 and the pump current Ip2 were obtained. For the sensor elements 101 of post-degradation 1 and post-degradation 2, a plurality of data associating the carbon dioxide concentration with the voltage V2 and the pump current Ip2 were obtained in the same manner. Using these data, the graph plotting the relationship between the carbon dioxide concentration and the corresponding voltage V2 is FIG. 8, the graph plotting the relationship between the carbon dioxide concentration and the corresponding pump current Ip2 is FIG. 9, and the graph plotting the relationship between the voltage V2 and the corresponding pump current Ip2 is FIG. 10.

As can be seen from FIG. 8, the voltage V2 takes a value corresponding to the carbon dioxide concentration in the measurement gas; specifically, it was confirmed that the higher the carbon dioxide concentration, the larger the absolute value tends to become. In addition, for any of the sensor elements 101 in the initial state, post-degradation 1, and post-degradation 2, the correspondence relationship between the voltage V2 and the carbon dioxide concentration is almost the same, and it was confirmed that the correspondence relationship does not change even if the second measurement electrode 44 is degraded. On the other hand, as can be seen from FIG. 9, although the pump current Ip2 tends to have a larger absolute value as the carbon dioxide concentration increases, it was confirmed that the correspondence relationship between the pump current Ip2 and the carbon dioxide concentration changes as the second measurement electrode 44 degrades. More specifically, it was confirmed that the greater the degree of degradation of the second measurement electrode 44, the smaller the absolute value of the pump current Ip2 corresponding to the same carbon dioxide concentration. It was also confirmed that, even when the degree of degradation is the same, the higher the carbon dioxide concentration, the larger the difference between the pump current Ip2 of the sensor element 101 in the initial state and the pump current Ip2 of the sensor element 101 after degradation. In this way, although both the voltage V2 and the pump current Ip2 take values corresponding to the carbon dioxide concentration in the measurement gas, it was confirmed that the pump current Ip2 is more likely than the voltage V2 to change its value under the influence of degradation of the second measurement electrode 44. Therefore, as can be seen from FIG. 10, when the second measurement electrode 44 degrades, the correspondence relationship between the voltage V2 and the pump current Ip2 changes. This is considered to be for the following reasons. First, the relationship between the voltage V2 (the electromotive force generated between the second measurement electrode 44 and the reference electrode 42) and the carbon monoxide concentration around the second measurement electrode 44 follows the Nernst electromotive force equation regardless of whether or not the second measurement electrode 44 is degraded, and therefore is considered to be hardly affected by the degradation of the second measurement electrode 44. In contrast, when the second measurement electrode 44 degrades, its ability to oxidize carbon monoxide decreases, making it difficult for the pump current Ip2 to flow, and it is considered that the absolute value of the pump current Ip2 corresponding to the same carbon dioxide concentration becomes smaller.

In the above-described degradation determination processing for the second measurement electrode 44, the degradation of the second measurement electrode 44 is determined by utilizing the fact that, as described above, the correspondence relationship between the voltage V2 and the pump current Ip2 changes when the second measurement electrode 44 degrades. Specifically, in step S250 of FIG. 6 described above, the CPU 97 applies the measured value of the voltage V2 to the correspondence relationship between the voltage V2 and the pump current Ip2 of the sensor element 101 in the initial state shown in FIG. 10, and takes the resulting pump current Ip2 as a converted pump current Ip2c. The correspondence relationship between the voltage V2 and the pump current Ip2 of the sensor element 101 in the initial state is stored in advance in the storage unit 98. Thus, the converted pump current Ip2c is derived as a value corresponding to the pump current Ip2 that would be obtained in step S240 in a case where the second measurement electrode 44 is not degraded. When, in actuality, the second measurement electrode 44 is degraded, the difference between this converted pump current Ip2c and the pump current Ip2 actually measured in step S240 becomes large, and therefore, in step S260, the CPU 97 determines the degradation of the second measurement electrode 44 based on this difference (in the present embodiment, based on the ratio Ip2c/Ip2).

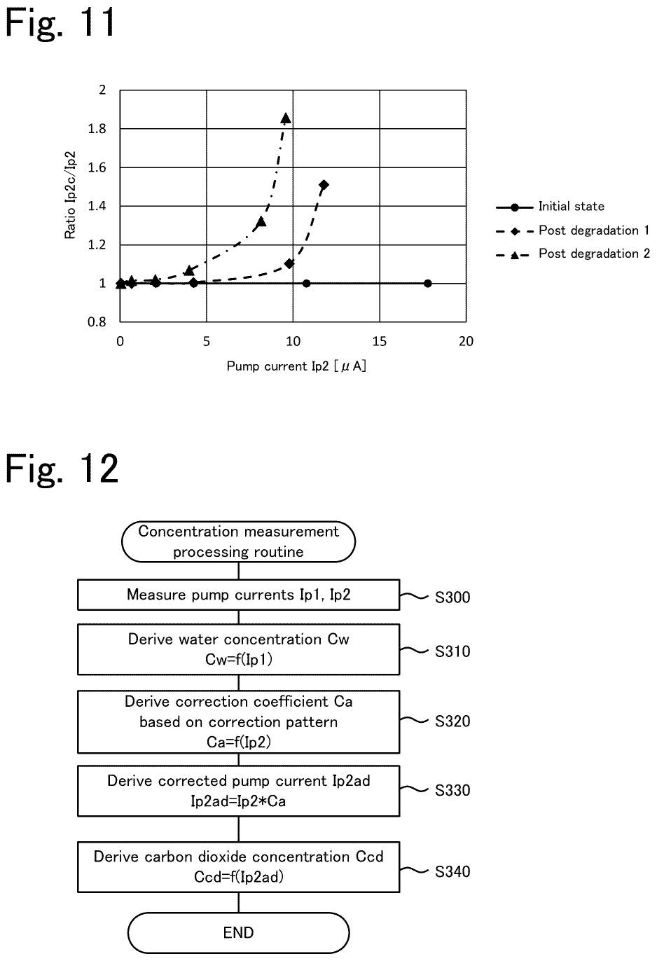

FIG. 11 is a graph showing a relationship between the measured value of the pump current Ip2 in step S240 and the ratio Ip2c/Ip2. FIG. 11 corresponds to a graph in which, for each point plotted in FIG. 10 (data associating a measured value of the voltage V2 with a measured value of the pump current Ip2), the value of the voltage V2 is converted into the converted pump current Ip2c, and further converted into the ratio Ip2c/Ip2, thereby transforming the data into data associating the ratio Ip2c/Ip2 with the measured value of the pump current Ip2. As can be seen from FIG. 11, in the sensor element 101 in the initial state, the ratio Ip2c/Ip2 is always approximately 1 regardless of the magnitude of the measured value of the pump current Ip2, whereas the larger the degree of degradation of the second measurement electrode 44, the more the ratio Ip2c/Ip2 tends to take a value greater than 1. Therefore, as described above, in step S260 the CPU 97 can determine the degradation of the second measurement electrode 44 by comparing the ratio Ip2c/Ip2 with thresholds Rref1 and Rref2. However, as can be seen from FIG. 11, even when the degree of degradation of the second measurement electrode 44 is the same, the ratio Ip2c/Ip2 increases as the measured value of the pump current Ip2 increases. Therefore, it is preferable that the thresholds Rref1 and Rref2 be values that tend to become larger as the measured value of the pump current Ip2 becomes larger (which can also be expressed as the converted pump current Ip2c becoming larger, or the carbon dioxide concentration in the measurement gas becoming larger). In this case, for example, as a curve or a polygonal line passing between the initial-state data (solid line) and the post-degradation-1 data (dashed line) in FIG. 11, the correspondence relationship between the measured value of the pump current Ip2 and the threshold Rref1 is predetermined and stored in the storage unit 98. Similarly, as a curve or a polygonal line passing between the post-degradation-1 data (dashed line) and the post-degradation-2 data (one-dot chain line) in FIG. 11, the correspondence relationship between the measured value of the pump current Ip2 and the threshold Rref2 is predetermined and stored in the storage unit 98. Then, in step S260, the CPU 97 applies the measured value of the pump current Ip2 in step S240 to these correspondence relationships stored in advance in the storage unit 98, derives the thresholds Rref1 and Rref2 corresponding to the measured value of the pump current Ip2, and uses them to determine the degradation of the second measurement electrode 44. In this way, the CPU 97 can appropriately determine, using the thresholds Rref1 and Rref2, to which of the initial state, post-degradation 1, and post-degradation 2 in FIG. 11 the degree of degradation of the second measurement electrode 44 is closer.

The ratio Ip2c/Ip2 can also be regarded as a correction coefficient for correcting the measured value of the pump current Ip2 to the converted pump current Ip2c (that is, to the value of the pump current Ip2 that would flow in a case where the second measurement electrode 44 is not degraded). Therefore, the correction patterns set in steps S270 to S290 of FIG. 6 can be predetermined based on the graph shown in FIG. 11. In the present embodiment, among the correction patterns of FIG. 7, the post-degradation pattern 1 is defined based on the post-degradation-1 data in FIG. 11, and the post-degradation pattern 2 is defined based on the post-degradation-2 data in FIG. 11. However, in the present embodiment, rather than directly adopting the post-degradation-1 and post-degradation-2 data of FIG. 11 as the post-degradation pattern 1 and the post-degradation pattern 2 of FIG. 7, the pump current Ip2 is divided into four regions as described above, and a correction pattern is used in which a single value of the correction coefficient Ca is associated with each region. That is, in the present embodiment, a simplified correction pattern is used that allows a certain degree of correction error.

Subsequently, an example of the concentration measurement processing will be described. FIG. 12 is a flowchart showing one example of a concentration measurement processing routine. This routine is stored, for example, in the storage unit 98 of the control unit 96 and is repeatedly executed by the CPU 97.

When the concentration measurement processing routine of FIG. 12 is executed, the CPU 97 first measures a pump current Ip1 flowing through the first measurement pump cell 50 by the first measurement pump control processing, and a pump current Ip2 flowing through the second measurement pump cell 41 by the second measurement pump control processing (step S300). Subsequently, the CPU 97 derives a water concentration Cw in the measurement gas based on the measured value of the pump current Ip1 (step S310). For example, the CPU 97 derives the water concentration Cw using a water concentration derivation map. The water concentration derivation map is stored in advance in the storage unit 98 as a correspondence relationship between the absolute value of the pump current Ip1 and the water concentration Cw, which is defined by experiments or analysis. In step S310, the CPU 97 applies the absolute value of the pump current Ip1 measured in step S300 to the water concentration derivation map, and derives the water concentration Cw corresponding to the absolute value of the pump current Ip1. In this manner, the water concentration in the measurement gas is measured.

Subsequently, the CPU 97 derives a correction coefficient Ca from the measured value of the pump current Ip2 input in step S300, based on the correction pattern set in accordance with the degradation determination result for the second measurement electrode 44 (steps S270 to S290 of FIG. 6) described above (step S320). That is the CPU 97 applies the measured value of the pump current Ip2 input in step S300 to the correction pattern currently set (the correction pattern most recently set) among the three types of correction patterns shown in FIG. 7 and derives the correction coefficient Ca corresponding to the measured value of the pump current Ip2. Then, the CPU 97 corrects the measured value of the pump current Ip2 input in step S300 using this correction coefficient Ca and derives a corrected pump current Ip2ad (step S330). Specifically, the CPU 97 derives the corrected pump current Ip2ad lby multiplying the measured value of the pump current Ip2 by the correction coefficient Ca.

After deriving the corrected pump current Ip2ad in step S330, the CPU 97 derives a carbon dioxide concentration Ccd in the measurement gas based on the corrected pump current Ip2ad (step S340), and terminates this routine. In step S340, for example, the CPU 97 derives the carbon dioxide concentration Ccd using a carbon dioxide concentration derivation map. The carbon dioxide concentration derivation map is stored in advance in the storage unit 98 as a correspondence relationship between the absolute value of the pump current Ip2 and the carbon dioxide concentration Ccd, which is defined by experiments or analysis. In step S340, the CPU 97 applies the corrected pump current Ip2ad derived in step S330 to this carbon dioxide concentration derivation map, and derives the carbon dioxide concentration Ccd corresponding to this value. When the second measurement electrode 44 degrades, the value of the pump current Ip2 corresponding to the carbon dioxide concentration decreases as shown in FIG. 9; however, by deriving the carbon dioxide concentration Ccd using the corrected pump current Ip2ad, the decreased pump current Ip2 can be corrected, and a decrease in the measurement accuracy of the carbon dioxide concentration Ccd due to degradation of the second measurement electrode 44 can be suppressed.

It is thought that specific modes of degradation of the first measurement electrode 51 and the second measurement electrode 44 is a decrease in the oxidation capability of the electrodes due to a reduction in active sites as catalysts caused by progress of sintering of the first type of noble metal contained in the electrodes. In addition, when the first measurement electrode 51 contains the second type of noble metal, it is also conceivable that, due to evaporation of this second type of noble metal, the three-phase interface of the noble metal, the solid electrolyte, and the measurement gas in the first measurement electrode 51 decreases, the resistance value of the first measurement electrode 51 increases, and the pump current Ip1 is less likely to flow (that is, the ability of the first measurement electrode 51 to oxidize hydrogen decreases).

Here, the correspondence relationship between the elements according to the present embodiment and the elements according to the present invention will be clarified. The sensor element 101 according to the present embodiment corresponds to the sensor element according to the invention. The control device 95 corresponds to the control device. The element body 102 corresponds to the element body. The first internal cavity 20 corresponds to the first chamber. The inner pump electrode 22 corresponds to the first inner electrode. The main pump cell 21 corresponds to the first pump cell. The second internal cavity 40 corresponds to the second chamber. The first measurement electrode 51 corresponds to the second inner electrode. The first measurement pump cell 50 corresponds to the second pump cell. The third internal cavity 61 corresponds to the third chamber. The second measurement electrode 44 corresponds to the third inner electrode. The second measurement pump cell 41 corresponds to the third pump cell. The outer pump electrode 23 corresponds to the first outer electrode, the second outer electrode, and the third outer electrode. The reference electrode 42 corresponds to the reference electrode. The main pump control processing corresponds to the first pump cell control processing. The first measurement pump control processing corresponds to the second pump cell control processing. The second measurement pump control processing corresponds to the third pump cell control processing. The pump current Ip1 corresponds to the second pump current. The pump current Ip2 corresponds to the third pump current. The voltage V2 corresponds to the third voltage. The corrected pump current Ip2ad corresponds to the corrected third pump current. Steps S300 and S320 to S340 in FIG. 12 correspond to the carbon dioxide concentration measurement processing. The degradation determination processing for the second measurement electrode 44 corresponds to the third inner electrode degradation determination processing. The degradation determination processing for the first measurement electrode 51 corresponds to the second inner electrode degradation determination processing.

According to the gas sensor 100 of the present embodiment described in detail above, the control device 95 performs degradation determination processing for the second measurement electrode 44 based on the voltage V2 between the second measurement electrode 44 and the reference electrode 42 during execution of the main pump control processing and the first measurement pump control processing and during stoppage of the second measurement pump control processing, and based on the pump current Ip2 flowing through the second measurement pump cell 41 during execution of the main pump control processing, the first measurement pump control processing, and the second measurement pump control processing. As described above, the pump current Ip2 is more likely than the voltage V2 to change its value under the influence of degradation of the second measurement electrode 44, and when the second measurement electrode 44 degrades, the correspondence relationship between the voltage V2 and the pump current Ip2 changes. Therefore, the degradation of the second measurement electrode 44 can be determined based on the voltage V2 and the pump current Ip2.

In the degradation determination processing for the second measurement electrode 44, the control device 95 measures the pump current Ip2 in step S240 after measuring the voltage V2 in step S220. Here, the pump current Ip2 has higher responsiveness than the voltage V2, that is, the time required for it to reach a value corresponding to the concentration of carbon monoxide in the third internal cavity 61 (a value corresponding to the carbon dioxide concentration in the measurement gas) is shorter. Therefore, by measuring the voltage V2 first and then measuring the pump current Ip2, the time difference between the two measurements can be reduced. As a result, it is possible to suppress a decrease in the accuracy of determining the degradation of the second measurement electrode 44 due to a change in the carbon dioxide concentration in the measurement gas occurring between the two measurements.

Furthermore, in the degradation determination processing for the second measurement electrode 44, the control device 95 determines the degradation of the second measurement electrode 44 based on the difference (here, the value of the ratio Ip2c/Ip2) between the converted pump current Ip2c derived from the measured value of the voltage V2 and the measured value of the pump current Ip2. In this way, the degradation of the second measurement electrode 44 can be determined relatively easily.

Furthermore, in the carbon dioxide concentration measurement processing, the control device 95 derives the carbon dioxide concentration Ccd based on the corrected pump current Ip2ad obtained by correcting the pump current Ip2 taking into account the result of the degradation determination processing for the second measurement electrode 44. Thus, by correcting the pump current Ip2, it is possible to suppress the decrease in the measurement accuracy of the carbon dioxide concentration Ccd due to degradation of the second measurement electrode 44.

Furthermore, in the carbon dioxide concentration measurement processing, the control device 95 performs the correction such that the greater the difference between the converted pump current Ip2c and the measured value of the pump current Ip2 in the degradation determination processing for the second measurement electrode 44 (here, the smaller the value of the ratio Ip2c/Ip2), the more the absolute value of the pump current Ip2 tends to be increased. More specifically, compared with the correction pattern (post-degradation pattern 1) for the case in which the value of the ratio Ip2c/Ip2 is greater than the threshold Rref1 and equal to or less than the threshold Rref2, a correction pattern (post-degradation pattern 2) is used in which a larger correction coefficient Ca than in post-degradation pattern 1 tends to be derived when the value of the ratio Ip2c/Ip2 is greater than the threshold Rref2. In this way, since more appropriate correction can be performed according to the degree of degradation of the second measurement electrode 44, it is possible to further suppress the decrease in the measurement accuracy of the carbon dioxide concentration Ccd due to degradation of the second measurement electrode 44.

Furthermore, the control device 95 performs degradation determination processing for the first measurement electrode 51 based on whether or not the absolute value of the voltage V2 during execution of the main pump control processing and the first measurement pump control processing and during stoppage of the second measurement pump control processing falls within the predetermined high-voltage region. When the control device 95 determines in the degradation determination processing for the first measurement electrode 51 that the first measurement electrode 51 is degraded (that is, when the first measurement electrode degradation flag is on), the control device 95 does not perform the determination of degradation of the second measurement electrode 44 in the degradation determination processing for the second measurement electrode 44. As described above, when the first measurement electrode 51 degrades, hydrogen reaches the second measurement electrode 44, whereby the absolute value of the voltage V2 increases; therefore, the degradation of the first measurement electrode 51 can be determined based on whether or not the absolute value of the voltage V2 falls within the predetermined high-voltage region. That is, in this gas sensor 100, not only the degradation of the second measurement electrode 44 but also the degradation of the first measurement electrode 51 can be determined. In addition, since the absolute value of the voltage V2 increases as described above when the first measurement electrode 51 is degraded, the correspondence relationship between the voltage V2 and the pump current Ip2 changes even if the second measurement electrode 44 is not degraded, with the result that the accuracy of determining the degradation of the second measurement electrode 44 may decrease. In contrast, in the gas sensor 100, the control device 95 does not perform the determination of degradation of the second measurement electrode 44 when it determines that the first measurement electrode 51 is degraded, and therefore the decrease in the accuracy of determining the degradation of the second measurement electrode 44 can be suppressed.

Furthermore, the measurement gas is exhaust gas of the internal combustion engine, and the control device 95 performs the degradation determination processing for the first measurement electrode 51 during fuel cut of the internal combustion engine or during stoppage thereof. Here, since the exhaust gas (gas in the exhaust pipe) during fuel cut or stoppage of the internal combustion engine has a lower carbon dioxide concentration than the exhaust gas during operation other than during fuel cut of the internal combustion engine, the influence of carbon monoxide reaching the third internal cavity 61 on the magnitude of the absolute value of the voltage V2 becomes small, and the influence of hydrogen becomes dominant. Therefore, the period during fuel cut or stoppage of the internal combustion engine is suitable timing for performing the degradation determination processing for the first measurement electrode 51.

It should be noted that the present invention is not limited to the present embodiment described above in any way, and it goes without saying that the present invention can be implemented in various modes as long as they fall within the technical scope of the present invention.

For example, in the embodiment described above, although the CPU 97 measures the pump current Ip2 in step S240 after measuring the voltage V2 in step S220, this order may be reversed. That is, the CPU 97 may perform steps S210 to S230 after measuring the pump current Ip2 in step S240. Further, regardless of whether or not these orders are reversed, the CPU 97 may perform step S220 after a predetermined waiting time Tw1 has elapsed since executing step S210. In addition, when the measurement of the voltage V2 is carried out first as in FIG. 6, that is, when the pump current Ip2 is measured in step S240 after the second measurement pump control processing is restarted in step S230 following the stoppage of the second measurement pump control processing, the CPU 97 may perform step S240 after a predetermined waiting time Tw2 has elapsed since executing step S230. The waiting time Tw1 can be predetermined in accordance with the time from stopping the second measurement pump control processing (stopping the pump current Ip2) until the voltage V2 reaches a value corresponding to the concentration of carbon monoxide in the third internal cavity 61 (a value corresponding to the carbon dioxide concentration in the measurement gas). The waiting time Tw2 can be predetermined in accordance with the time from restarting the second measurement pump control processing until the pump current Ip2 reaches a value corresponding to the concentration of carbon monoxide in the third internal cavity 61 (a value corresponding to the carbon dioxide concentration in the measurement gas). Both of the waiting times Tw1 and Tw2 can be, for example, on the order of several milliseconds to a dozen-odd milliseconds. Further, as described above, since the pump current Ip2 has higher responsiveness than the voltage V2, it is possible to set Tw2<Tw1. When the voltage V2 is measured first, the waiting time Tw2 is included in the time difference between the measurement of the voltage V2 and the measurement of the pump current Ip2. When the pump current Ip2 is measured first, the waiting time Tw1 is included in the time difference between the measurement of the voltage V2 and the measurement of the pump current Ip2. Note that only the waiting time Tw1 may be set with the waiting time Tw2 set to zero, or both the waiting times Tw1 and Tw2 may be set to zero. The CPU 97 may also wait for the lapse of the waiting time Tw1 between step S110 and step S120.

In the embodiment described above, when the absolute value of the voltage V2 measured in step S220 falls within a predetermined low-voltage region, the CPU 97 may refrain from performing the determination of degradation of the second measurement electrode 44. As can be seen from FIGS. 9 and 10, when the absolute value of the voltage V2 is relatively low, in other words, when the carbon dioxide concentration in the measurement gas is relatively low, the value of the pump current Ip2 is less likely to change among the initial state and post-degradation 1 and 2. That is, it is difficult to determine whether or not the second measurement electrode 44 has degraded. Therefore, for example, based on FIG. 10, the upper limit of the low-voltage region is set to 850 mV, and when the absolute value of the voltage V2 measured in step S220 is 850 mV or less, the CPU 97 may terminate the second processing routine without performing the processing subsequent to step S240. In this way, by refraining from determining the degradation of the second measurement electrode 44 when the absolute value of the voltage V2 falls within the predetermined low-voltage region, the processing load of the control device 95 can be reduced. Moreover, it is also possible to suppress a case where, when the second measurement electrode 44 is degraded, the CPU 97 performs an erroneous determination that the second measurement electrode 44 is not degraded and sets the correction pattern to the initial pattern.

In the embodiment described above, the degradation of the second measurement electrode 44 was determined based on the difference between the converted pump current Ip2c derived from the measured value of the voltage V2 and the measured value of the pump current Ip2, however, the present invention is not limited thereto. When comparing the measured value of the voltage V2 and the measured value of the pump current Ip2 for determining the degradation of the second measurement electrode 44, at least one of the two measured values may be converted into any one of the voltage V2, the pump current Ip2, or the carbon dioxide concentration for comparison. For example, the CPU 97 may determine the degradation of the second measurement electrode 44 based on a difference between a carbon dioxide concentration derived from the measured value of the voltage V2 and a carbon dioxide concentration derived from the measured value of the pump current Ip2. The measured value of the voltage V2 can be converted into the carbon dioxide concentration based on, for example, the correspondence relationship represented by the initial-state data in FIG. 8, and the measured value of the pump current Ip2 can be converted into the carbon dioxide concentration based on, for example, the correspondence relationship represented by the initial-state data in FIG. 9. Then, if the pump current Ip2 has decreased due to degradation of the second measurement electrode 44, the carbon dioxide concentration converted from the voltage V2 and the carbon dioxide concentration converted from the pump current Ip2 will not match, and the latter will be derived as a lower value. Therefore, similarly to the embodiment described above, the CPU 97 can determine the degradation of the second measurement electrode 44 based on a difference between the carbon dioxide concentrations obtained by converting the two measured values. Alternatively, the CPU 97 may determine the degradation of the second measurement electrode 44 based on a difference between the measured value of the voltage V2 and a voltage V2 derived from the measured value of the pump current Ip2. The measured value of the pump current Ip2 can be converted into the voltage V2 based on, for example, the correspondence relationship represented by the initial-state data in FIG. 10. Then, if the pump current Ip2 has decreased due to degradation of the second measurement electrode 44, the measured value of the voltage V2 and the voltage V2 converted from the pump current Ip2 will not match, and the latter will be derived as a lower value. Therefore, similarly to the embodiment described above, the CPU 97 can determine the degradation of the second measurement electrode 44 based on a difference between the measured value of the voltage V2 and the voltage V2 converted from the measured value of the pump current Ip2. Alternatively, the CPU 97 may determine the degradation of the second measurement electrode 44 based on the measured value of the voltage V2 and the measured value of the pump current Ip2 without performing such conversions. For example, a band-like region including the curve represented by the initial-state data in FIG. 10 and its vicinity is set as a region representing the correspondence relationship to be satisfied between the voltage V2 and the pump current Ip2 when there is no degradation in the second measurement electrode 44 (a region that can be regarded as absence of degradation in the second measurement electrode 44), and information such as inequalities or a map representing that region is stored in advance in the storage unit 98. Then, the CPU 97 may determine whether or not the second measurement electrode 44 is degraded depending on whether the correspondence relationship between the measured value of the voltage V2 and the measured value of the pump current Ip2 falls outside the region stored in the storage unit 98.

In the embodiment described above, when deriving the carbon dioxide concentration Ccd, the CPU 97 performed a correction taking into account the result of the degradation determination for the second measurement electrode 44; however, instead of or in addition to performing the correction, when it is determined in step S260 that degradation has occurred in the second measurement electrode 44, an abnormality of the gas sensor 100 may be notified to another device such as the engine ECU or to a user such as the driver.

In the embodiment described above, the CPU 97 made a three-level determination of degradation (no degradation, slight degradation, and significant degradation) for the second measurement electrode 44 using thresholds Rref1 and Rref2, however, the invention is not limited thereto, and it is also possible to determine only whether degradation has occurred or not, or to perform a determination of four or more levels according to the degree of degradation. Similarly, in the embodiment described above, the CPU 97 used three correction patterns shown in FIG. 7 according to the degree of degradation of the second measurement electrode 44, however, the invention is not limited thereto, and it is also possible to use two correction patterns for the case where degradation has occurred and the case where degradation has not occurred, or to use four or more correction patterns according to the degree of degradation. In addition, in the embodiment described above, although the CPU 97 used correction patterns created by simplifying the post-degradation-1 and post-degradation-2 data of FIG. 11, however, the invention is not limited thereto. For example, using the post-degradation-1 and post-degradation-2 data, and data interpolating an intermediate degraded state between the two, a formula or a map describing the correspondence relationship between the measured value of the pump current Ip2 and the correction coefficient Ca may be predetermined and stored in the storage unit 98 and used as a correction pattern. Moreover, in the embodiment described above, in the correction pattern the measured value of the pump current Ip2 was associated with the correction coefficient Ca, but the measured value of the pump current Ip2 may be associated with the corrected pump current Ip2ad.

In the embodiment described above, the CPU 97 derived a corrected pump current Ip2ad, obtained by performing a correction on the measured value of the pump current Ip2 in consideration of the result of the degradation determination processing for the second measurement electrode 44, and derived the carbon dioxide concentration Ccd based on the corrected pump current Ip2ad, however, the invention is not limited thereto. For example, the CPU 97 may derive a provisional carbon dioxide concentration Ccdt based on the measured value of the pump current Ip2, and derive a carbon dioxide concentration Ccd obtained by performing a correction on the provisional carbon dioxide concentration Ccdt in consideration of the result of the degradation determination processing for the second measurement electrode 44. Even in this case, as in the embodiment described above, since the carbon dioxide concentration Ccd is derived by taking into account the result of the degradation determination processing for the second measurement electrode 44 with respect to the measured value of the pump current Ip2, a decrease in the measurement accuracy of the carbon dioxide concentration Ccd due to degradation of the second measurement electrode 44 can be suppressed. For example, when deriving the provisional carbon dioxide concentration Ccdt based on the measured value of the pump current Ip2, the CPU 97 may derive, as the provisional carbon dioxide concentration Ccdt, the carbon dioxide concentration obtained by applying the measured value of the pump current Ip2 to the carbon dioxide concentration derivation map used in step S340. Further, when deriving the carbon dioxide concentration Ccd by performing a correction on the provisional carbon dioxide concentration Ccdt in consideration of the result of the degradation determination processing for the second measurement electrode 44, the CPU 97 may perform a correction such that the provisional carbon dioxide concentration Ccdt tends to be increased to a greater extent as the degree of degradation of the second measurement electrode 44 becomes larger (for example, as the ratio Ip2c/Ip2 becomes larger, that is, as the difference between the measured value of the pump current Ip2 and the converted pump current Ip2c becomes larger), and derive the carbon dioxide concentration Ccd. This correction may be performed, for example, as follows. First, similarly to the correction patterns in the embodiment described above, a plurality of types of correction patterns indicating the relationship between the provisional carbon dioxide concentration Ccdt and a correction coefficient are prepared according to the presence or absence and the degree of degradation of the second measurement electrode 44 and stored in the storage unit 98. Then, among the plurality of correction patterns, the CPU 97 uses the correction pattern set based on the result of the degradation determination processing for the second measurement electrode 44 to derive a correction coefficient corresponding to the provisional carbon dioxide concentration Ccdt, and derives the carbon dioxide concentration Ccd by multiplying the provisional carbon dioxide concentration Ccdt by the correction coefficient to correct the provisional carbon dioxide concentration Ccdt.

In the embodiment described above, when the operating state of the internal combustion engine differs between the time of measurement of the voltage V2 in step S220 and the time of measurement of the pump current Ip2 in step S240, the CPU 97 may be configured not to determine the degradation of the second measurement electrode 44. Here, when the operating state of the internal combustion engine differs, the carbon dioxide concentration in the measurement gas is also likely to differ. If the carbon dioxide concentration differs between the time of measurement of the voltage V2 and the time of measurement of the pump current Ip2, the correspondence relationship between the voltage V2 and the pump current Ip2 changes due to factors other than degradation of the second measurement electrode 44, with the result that the accuracy of determining the degradation of the second measurement electrode 44 may decrease. Therefore, by refraining from determining the degradation of the second measurement electrode 44 when the operating state of the internal combustion engine differs between the time of measurement of the voltage V2 and the time of measurement of the pump current Ip2, it is possible to suppress a decrease in the accuracy of determining the degradation of the second measurement electrode 44. For example, the CPU 97 may, after step S240, determine whether the operating state of the internal combustion engine has changed between step S220 and step S240, and, if it is determined that the operating state has changed, terminate the second processing routine without performing the processing subsequent to step S250. The CPU 97 may periodically acquire information representing the operating state of the internal combustion engine, such as information regarding the air-fuel ratio of the internal combustion engine or information regarding the fuel injection amount of the internal combustion engine, from another device (for example, an engine ECU or an air-fuel ratio sensor), and detect a change in the operating state of the internal combustion engine based on the acquired information. Alternatively, the CPU 97 may be configured to detect the oxygen concentration around the sensor element 101 based on the voltage Vref of the sensor cell 83 and detect the change in the operating state of the internal combustion engine based on a change in the oxygen concentration detected.

In the embodiment described above, the CPU 97 performed the degradation determination processing for the first measurement electrode 51 when the internal combustion engine was in fuel cut or stopped, however, the invention is not limited thereto. As shown in FIG. 5, since the value of the voltage V2 caused by carbon monoxide around the second measurement electrode 44 differs from the value of the voltage V2 caused by hydrogen, it is possible to determine whether hydrogen is present around the second measurement electrode 44 even when carbon monoxide is present around the second measurement electrode 44, for example, based on the measured value of the voltage V2 and the threshold V2ref1. Therefore, the CPU 97 can perform the degradation determination processing for the first measurement electrode 51 not only during fuel cut (a timing when the carbon dioxide concentration in the measurement gas is low). For example, in place of step S100, the CPU 97 may determine, in the current use of the gas sensor 100, whether or not the degradation determination processing for the first measurement electrode 51 has not been carried out or a predetermined time T1 has elapsed since the previous execution, and perform the processing subsequent to step S110 when it has not been carried out or when the predetermined time T1 has elapsed since the previous execution. The predetermined time T1 may be set, for example, to a time until the possibility arises that the first measurement electrode 51 will degrade from the initial state, or to a shorter time, and may be, for example, on the order of several seconds to several minutes, or on the order of 1000 hours to several thousand hours.

In the embodiment described above, the CPU 97 may be configured not to execute the first processing routine, that is, not to perform the degradation determination processing for the first measurement electrode 51.

In the embodiment described above, in the second processing routine, when the first measurement electrode degradation flag was on in step S205, the degradation determination of the second measurement electrode 44 was not performed and the second routine was terminated. In addition to or instead of this, the CPU 97 may perform the same determination as step S130 of the first processing routine on the measured value of the voltage V2 in step S220. That is, the CPU 97 may determine whether or not the absolute value of the voltage V2 measured in step S220 falls within a predetermined high-voltage region. In the case of an affirmative determination, the CPU 97 may terminate the second processing routine without performing the processing subsequent to step S240. Also in this case, since the CPU 97 is prevented from determining the degradation of the second measurement electrode 44 when the first measurement electrode 51 is degraded, a decrease in the accuracy of determining the degradation of the second measurement electrode 44 can be suppressed.

In the embodiment described above, the CPU 97 may be configured not to perform step S310, that is, not to perform the measurement of the water concentration.

In the above-described embodiment, the outer pump electrode 23 plays a role as the first outer electrode to be paired with the inner pump electrode 22 of the main pump cell 21, a role as the second outer electrode to be paired with the first measurement electrode 51 of the first measurement pump cell 50, and a role as the third outer electrode to be paired with the second measurement electrode 44 of the second measurement pump cell 41. In other words, the first to third outer electrodes are configured as a common outer pump electrode 23. However, the present invention is not limited thereto. For example, two of the first to third outer electrodes may be configured as the common outer pump electrode, and the remaining one may be disposed on an outer surface of the element body 102 as an electrode independent of the outer pump electrode 23 so as to be in contact with the measurement gas. Alternatively, the first to third outer electrodes may each be disposed on an outer surface of the element body 102 as independent electrodes so as to be in contact with the measurement gas.

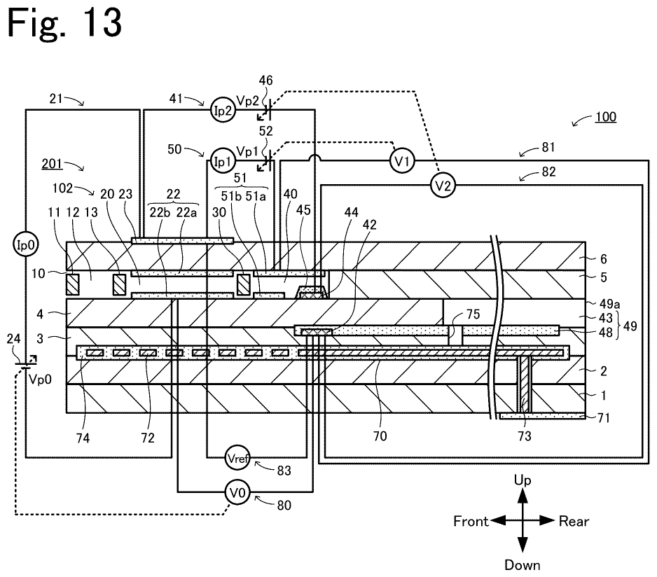

In the above-described embodiment, the sensor element 101 of the gas sensor 100 includes the first internal cavity 20, the second internal cavity 40, and the third internal cavity 61. However, the present invention is not limited thereto. For example, as shown in FIG. 13, a sensor element 201 according to a modification may not include the third internal cavity 61. In the sensor element 201 according to a modification shown in FIG. 13, between the lower surface of the second solid electrolyte layer 6 and the upper surface of the first solid electrolyte layer 4, the following components are adjacently formed and in communication with each other, in the order listed: a gas inlet 10; a first diffusion rate-limiting section 11; a buffer space 12; a second diffusion rate-limiting section 13; a first internal cavity 20; a third diffusion rate-limiting section 30; and a second internal cavity 40. In addition, the second measurement electrode 44 is disposed on the upper surface of the first solid electrolyte layer 4 within the second internal cavity 40. The second measurement electrode 44 is covered with a fourth diffusion rate-limiting section 45, which is a film made of a porous ceramic material such as alumina (Al₂O₃). The fourth diffusion rate-controlling section 45, similarly to the fourth diffusion rate-controlling section 60 of the above-described embodiment, serves to impart a predetermined diffusion resistance to the measurement gas in the second internal cavity 40 and guide the measurement gas to the second measurement electrode 44. Furthermore, the fourth diffusion rate-limiting section 45 also functions as a protective film for the second measurement electrode 44. The ceiling electrode portion 51a of the first measurement electrode 51 extends directly above the second measurement electrode 44. Even with such a configuration of the sensor element 201, the carbon dioxide concentration Ccd can be measured based on the pump current Ip2 flowing through the second measurement pump cell 41, similarly to the embodiment described above. In the sensor element 201 of FIG. 13, the region around the second measurement electrode 44 functions as the third chamber. That is, the area around the second measurement electrode 44 serves the same role as the third internal cavity 61.

In the above-described embodiment, the element body 102 of the sensor element 101 is formed as the laminated body including multiple solid electrolyte layers (layers 1 to 6). However, the present invention is not limited thereto. The element body of the sensor element 101 only needs to include at least one oxygen ion-conductive solid electrolyte layer, and have a measurement gas flow path therein. For example, in FIG. 1, layers 1 to 5, except for the second solid electrolyte layer 6, may be structural layers made of materials other than solid electrolyte (e.g., layers made of alumina). In such a case, each electrode included in the sensor element 101 may be disposed on the second solid electrolyte layer 6. For instance, the second measurement electrode 44 shown in FIG. 1 may be disposed on a lower surface of the second solid electrolyte layer 6. Furthermore, the reference gas introduction space 43, which is formed in the first solid electrolyte layer 4, may instead be formed in the spacer layer 5. Likewise, the reference gas introduction layer 48, located between the first solid electrolyte layer 4 and the third substrate layer 3, may instead be provided between the second solid electrolyte layer 6 and the spacer layer 5. In addition, the reference electrode 42 may be provided on the lower surface of the second solid electrolyte layer 6 at a position downstream of the third internal cavity 61.

What is claimed is:

1. A gas sensor comprising a sensor element and a control device, and configured to measure a carbon dioxide concentration in a measurement gas,
wherein:
the sensor element includes:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on an outer surface of the element body;
a second pump cell including a second inner electrode disposed in a second chamber located downstream of the first chamber of the measurement gas flow path, and a second outer electrode disposed on an outer surface of the element body;
a third pump cell including a third inner electrode disposed in a third chamber located downstream of the second chamber of the measurement gas flow path, and a third outer electrode disposed on an outer surface of the element body; and
a reference electrode disposed inside the element body so as to be in contact with a reference gas;
the control device performs:
a first pump cell control processing in which oxygen is pumped out from around the first inner electrode to around the first outer electrode by controlling the first pump cell, thereby reducing water and carbon dioxide in the measurement gas in the first chamber;
a second pump cell control processing in which oxygen is pumped from around the second outer electrode to around the second inner electrode by controlling the second pump cell, thereby oxidizing hydrogen generated by the reduction of water in the first chamber in the second chamber;
a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and
a carbon dioxide concentration measurement processing for measuring the carbon dioxide concentration in the measurement gas based on a third pump current flowing through the third pump cell by the third pump cell control processing;
the control device performs a third inner electrode degradation determination processing for determining degradation of the third inner electrode based on a third voltage between the third inner electrode and the reference electrode during execution of the first and second pump cell control processing and during stoppage of the third pump cell control processing, and based on the third pump current flowing through the third pump cell during execution of the first to third pump cell control processing.

2. The gas sensor according to claim 1,
wherein the control device, in the third inner electrode degradation determination processing, measures the third voltage and thereafter measures the third pump current.

3. The gas sensor according to claim 1,
wherein the control device, in the third inner electrode degradation determination processing, does not perform a determination of degradation of the third inner electrode when the absolute value of the third voltage falls within a predetermined low-voltage region.

4. The gas sensor according to claim 1,
wherein the control device, in the third inner electrode degradation determination processing,
performs a determination of degradation of the third inner electrode based on a difference between the third pump current derived from a measured value of the third voltage and a measured value of the third pump current; or
performs a determination of degradation of the third inner electrode based on a difference between a carbon dioxide concentration derived from a measured value of the third voltage and a carbon dioxide concentration derived from a measured value of the third pump current; or
performs a determination of degradation of the third inner electrode based on a difference between a measured value of the third voltage and the third voltage derived from a measured value of the third pump current.

5. The gas sensor according to claim 1,
wherein the control device, in the carbon dioxide concentration measurement processing, derives the carbon dioxide concentration based on a corrected third pump current obtained by correcting the third pump current taking into account a result of the third inner electrode deterioration determination processing, or derives the carbon dioxide concentration by correcting a provisional carbon dioxide concentration based on the third pump current taking into account a result of the third inner electrode deterioration determination processing.

6. The gas sensor according to claim 4,
wherein the control device, in the carbon dioxide concentration measurement processing, derives the carbon dioxide concentration based on a corrected third pump current obtained by correcting the third pump current taking into account a result of the third inner electrode deterioration determination processing, or derives the carbon dioxide concentration by correcting a provisional carbon dioxide concentration based on the third pump current taking into account a result of the third inner electrode deterioration determination processing, and
the control device, in the carbon dioxide concentration measurement processing, performs a correction such that the greater the difference in the third inner electrode degradation determination processing, the more the absolute value of the third pump current or the provisional carbon dioxide concentration tends to be increased.

7. The gas sensor according to claim 1,
wherein the measurement gas is exhaust gas of an internal combustion engine, and
the control device, in the third inner electrode degradation determination processing, does not perform a determination of degradation of the third inner electrode when an operating state of the internal combustion engine differs between the time of measurement of the third voltage and the time of measurement of the third pump current.

8. The gas sensor according to claim 1,
wherein the control device, in the third inner electrode degradation determination processing, does not perform a determination of degradation of the third inner electrode when an absolute value of the third voltage falls within a predetermined high-voltage region.

9. The gas sensor according to claim 1,
wherein the control device performs a second inner electrode degradation determination processing for determining degradation of the second inner electrode based on whether or not an absolute value of the third voltage during execution of the first and second pump cell control processing and during stoppage of the third pump cell control processing falls within a predetermined high-voltage region, and
when the control device determines in the second inner electrode degradation determination processing that the second inner electrode is degraded, the control device does not perform a determination of degradation of the third inner electrode in the third inner electrode degradation determination processing.

10. The gas sensor according to claim 9,
wherein the measurement gas is exhaust gas of an internal combustion engine, and
the control device performs the second inner electrode degradation determination processing during fuel cut of the internal combustion engine or during stoppage thereof.

11. The gas sensor according to claim 1,
wherein the control device measures a water concentration in the measurement gas based on the second pump current flowing through the second pump cell by the second pump cell control processing.
